(19) United States
(12) Patent Application Publication
(10) Pub. No.: US 2011/0295255 A1
(43) Pub. Date: Dec. 1, 2011

(54) PROXIMAL FEMUR FIXATION APPARATUS, SYSTEMS AND METHODS WITH ANGLED ELONGATE ELEMENTS

(75) Inventors: Jeffrey Roberts, Germantown, TN (US); Thomas B. Buford, III, Memphis, TN (US); Michael Veldman, Memphis, TN (US); Terrance Strohkirch, Memphis, TN (US); Edward Perez, Memphis, TN (US); Jesse Moore, Germantown, TN (US)

(73) Assignee: SONOMA ORTHOPEDIC PRODUCTS, INC., Santa Rosa, CA (US)

(21) Appl. No.: 13/147,789

(22) PCT Filed: Feb. 5, 2010
(86) PCT No.: PCT/US10/23304

§ 371 Date: Aug. 3, 2011

Related U.S. Application Data
(60) Provisional application No. 61/150,134, filed on Feb. 5, 2009.

(51) Int. Cl. A61B 17/56

(52) U.S. Cl. 606/64

(57) ABSTRACT

An implantable femoral fixation device adapted to be received the femoral neck and femoral head configured to support at least a portion of the inner surface of the cortical bone of the femur. In various embodiments the femoral fixation device is transformable between a first radially reduced configuration and a second, radially expanded configuration, is attached to an anchor, and/or is configured to be encased in fixation media. In one embodiment the anchor is a segmented intramedullary structure disposed in the intramedullary canal configurable between a relatively flexible, bent configuration for implantation or extraction and a relatively rigid, straightened configuration for bone treatment.

### CROSS-REFERENCE TO RELATED APPLICATIONS

This application claims the benefit of priority of U.S. Provisional Application No. 61/150,134, filed Feb. 5, 2009.

This application incorporates by reference, in their entireties, all of the following applications: U.S. Provisional No. 61/150,134, filed Feb. 5, 2009, U.S. Provisional No. 61/180,342, filed May 21, 2009, U.S. application Ser. Nos. 12/345,451, 12/345,225 and 12/345,340 (all of which were filed Dec. 29, 2008 as continuations-in-part of U.S. application Ser. No. 12/052,919, filed Mar. 21, 2008), and U.S. application Ser. No. 12/052,919, filed Mar. 21, 2008, which claim the benefit of priority from U.S. Provisional No. 60/896,342 filed Mar. 22, 2007.

### BACKGROUND

There is a significant unmet clinical need for better devices and methods for treatment of proximal femoral fractures. Conventional technologies include various uses of screws, cannulated screws, compression hip screws, plates in trochanteric or interochanteric implants for treating femoral head and/or intertrochanteric fractures. Among patients treated with conventional technologies, studies have found that only 40% regain their pre-operative mobility, and only 24% regain the pre-operative function. The risk of these types of fractures increases in osteoporotic bone. As populations get older the incidence of these types of hip fractures will continue to increase.

One problem associated with soft, osteoporotic bone as is commonly observed in the elderly is a loss of mobility due to changes in the femoral head location. Conventional treatment of proximal femoral fractures with conventional devices can often lead to the use of hard metal structures such as nails or screws placed inside weak, osteoporotic bone. Even if an initial fusion or treatment is successful in fusing some or most of the bone segments after a fracture, over time, conventional implants may start to migrate with respect to the femoral head. In certain instances, an implant might stay in place with an anchor mechanism while the surrounding soft, cancellous or osteoporotic bone inside or near the femoral head is unable to support the implant. Convention implants can carve out a cavity or a path inside the soft or osteoporotic bone that leads to migration of implant, the femoral head, or both. Conventional attempts to fix this problem with the addition of supplemental screws or other plates and structures tend to increase damage in the bone, resulting in more bone loss and more significant potential for migration and injury.

### SUMMARY

Embodiments of the present invention relate to an orthopedic prosthesis, and, more particularly, to an implantable structure for fixation of proximal femoral fractures. In various embodiments a proximal femoral implant is configured to be used alone or in combination with an anchor. In some embodiments the anchor is an intramedullary structure. In some embodiments the anchor is an intramedullary (IM) nail. In various embodiments a proximal femoral implant is configured to be used with an intramedullary or IM nail that is adapted to be received in the intramedullary canal. In various embodiments the IM nail can be used to anchor the proximal femoral implant, fixate long bone fractures, or any combination of bone treatments.

In one embodiment, an IM nail is inserted through the greater trochanter in order to access the intramedullary canal in the femur. In one embodiment the IM nail is rigid. In some embodiments the IM nail can have a rigid configuration and a flexible or bending configuration. In one embodiment is a segmented intramedullary structure. In one embodiment a segmented intramedullary structure comprises a plurality of segments, each segment having a first interface and a complementarily-shaped second interface such that the first interface of a segment cooperatively engages the second interface of an adjacent segment, each segment including a channel. In one embodiment an elongate element extends through the channels to apply a compressive force along the longitudinal axis of the structure. In one embodiment a lock is disposed in at least one of the proximal end and the distal end for securing the tension member. In one embodiment, activation of the tensioning member causes the fixation structure to convert from a substantially flexible state to a substantially rigid state. In various embodiments, the IM nail can be any of the embodiments disclosed in U.S. Provisional No. 61/150,134, filed Feb. 5, 2009, U.S. Provisional No. 61/180,342, filed May 21, 2009, or any of U.S. application Ser. Nos. 12/345,451, 12/345,225 and 12/345,340 (all of which were filed Dec. 29, 2008 as continuations-in-part of U.S. application Ser. No. 12/052,919, filed Mar. 21, 2008), all of which are incorporated by reference, in their entireties herein. Use of embodiments of a segmented intramedullary structure can have the advantage of providing for extra-capsular entry points that do significantly less damage to surrounding tissue. For example, in one embodiment a segmented intramedullary structure can be used to provide an anchor for a proximal femoral implant. In one embodiment, a segmented intramedullary structure can be implanted with more lateral insertion than conventional access points. In one embodiment a single lateral incision can be made to insert a segmented intramedullary structure and to insert a proximal femoral implant through the same lateral incision point. In various embodiments, embodiments of a proximal femoral implant can be used alone, with an anchoring device, and/or with a segmented intramedullary structure.

In one embodiment, a method of treating a fracture in a proximal femur includes the steps of creating an access hole in cortical bone, creating a pathway in cancellous bone through the femoral neck and into the femoral head, creating a cavity in cancellous bone between the cavity and a portion of the inside surface of cortical bone in the femoral head, inserting a femoral fixation device through the access hole, inserting a fixation media to fill at least a portion of the cavity, and anchoring the femoral fixation device to an anchor. In one embodiment the method also includes transforming the femoral fixation device from a radially reduced configuration to a radially expanded configuration. In one embodiment the method also includes providing an anchor having a proximal end and a distal end, advancing the anchor along a nonlinear path while the anchor is in a flexible state, engaging the bone with the distal end of the anchor, transforming the anchor from the flexible state to a substantially rigid state, and locking the anchor in the substantially rigid state.

In one embodiment, a method of treating a fracture in a proximal femur includes the steps of creating an access hole in cortical bone, boring a cavity in cancellous bone through the femoral neck and into the femoral head, removing cancellous bone between the cavity and a portion of the inside surface of cortical bone in the femoral head, inserting a femoral fixation device through the access hole, inserting fixation media to fill at least a portion of the cavity, and anchoring the femoral fixation device to an anchor. In one embodiment the method also includes transforming the femoral fixation device from a radially reduced configuration to a radially expanded configuration. In one embodiment the method also includes providing an anchor having a proximal end and a distal end, advancing the anchor along a nonlinear path while the anchor is in a flexible state, engaging the bone with the distal end of the anchor, transforming the anchor from the flexible state to a substantially rigid state, and locking the anchor in the substantially rigid state.

In one embodiment, an implantable femoral fixation device includes a proximal segment, a distal segment and an intermediate segment disposed between the proximal segment and the distal segment. The implantable femoral fixation device includes a first configuration and a second configuration, the second configuration being larger than the first configuration and an anchor. In one embodiment the anchor comprising a segmented intramedullary structure. In one embodiment the anchor includes a plurality of segments, each segment having a first interface and a complementarily-shaped second interface such that the first interface of a segment cooperatively engages the second interface of an adjacent segment. In one embodiment each segment includes a channel. In one embodiment the anchor includes an elongate element extending through the channels to apply a compressive force along the longitudinal axis of the structure. In one embodiment the anchor includes a lock in at least one of the proximal end and the distal end, for securing the tension member, wherein activation of the tensioning member causes the fixation structure to convert from a substantially flexible state to a substantially rigid state. In one embodiment the anchor includes an elongate body, transformable between a flexible state for implantation within a bone, and a rigid state for fixing a fracture in a bone and a plurality of segments for defining the body. Each segment has a first interface and a complementarily-shaped second interface such that the first interface of a segment cooperatively engages the second interface of an adjacent segment, the segments comprising a channel so as to be receivable over a guide for positioning in the intramedullary canal, wherein the body is bendable in a single plane within the flexible state. In one embodiment the anchor includes a proximal end, a distal end and an elongate body adapted to be received in the intramedullary canal of a long bone, the anchor further comprising a plurality of segments. Each segment has a first interface and a complementarily-shaped second interface such that the first interface of a segment cooperatively engages the second interface of an adjacent segment, the segments including a guide lumen so as to be receivable over a guide for positioning in the intramedullary canal. In one embodiment the anchor includes a tensioning member extending through the fixation structure to apply a compressive force along the longitudinal axis of the structure. In one embodiment the anchor includes a lock in at least one of the proximal end and the distal end, for securing the tension member, wherein activation of the tensioning member causes the fixation structure to convert from a substantially flexible state to a substantially rigid state.

Various embodiments of a femur fixation structure are contemplated. In one embodiment the fixation structure comprises of a head portion, an intramedullary structure, and two or more elongate bodies. In one embodiment, the intramedullary structure and the elongate bodies couple or are attachable to the head portion. In one embodiment, the intramedullary structure and the elongate bodies couple to the head portion at such angles that the intramedullary structure can be disposed in the femoral intramedullary canal and the elongate bodies can be disposed in the femoral neck and femoral head. In one embodiment the elongate bodies comprise a proximal end and a distal end. In one embodiment, in the course of coupling to the head portion, the elongate bodies define two positions: converged and diverged. In one embodiment, the total distance between the distal ends in the converged position defines a first distance, and the total distance between the distal ends in the diverged position defines a second distance. In one embodiment, the second distance is greater than the first distance. In one embodiment, at least one elongate body preferably comprises a threaded section, which can couple to osseous tissue.

In one embodiment the intramedullary structure comprises a base, a distal portion, and a middle portion. In one embodiment, the intramedullary structure is configured to be receivable within an intramedullary passage. In one embodiment the intramedullary structure is straight, while in other embodiments the intramedullary structure is angled or curved. For example, in one embodiment, the middle portion of the structure is curved. In one embodiment the structure comprises a plurality of interconnecting segments, such as is disclosed in U.S. patent application No. 61/150134, filed Feb. 5, 2009, which is incorporated by reference in its entirety herein. In one embodiment, the intramedullary structure is constructed from a biocompatible material, for example but not limited to, titanium, stainless steel, tungsten, polymer, polyether ether ketone (PEEK), or the like.

In one embodiment, the elongate bodies comprise a proximal end that is coupleable to the anchor and a distal end configured to be positioned in a bony structure, such as the femoral head and/or femoral neck. In one embodiment the elongate bodies have threads to attach to threaded apertures in the anchor. In one embodiment, the elongate bodies attach to the anchor in such an angle that the distal ends of the bodies diverge from each other. In other words, the distance between the distal ends of the elongate bodies is preferably greater than the distance between the proximal ends of the elongate bodies. In various embodiments the elongate bodies are of the same diameter, disparate diameters, or combinations thereof. In some embodiments some or all of the elongate members are the same length, but in other embodiment some or all of the elongate bodies are of different lengths. In some embodiments, some or all of the elongate bodies are the same type, such as a bolt, screw, rod, molly-bolt, or the like. In some embodiments, all the elongate bodies are of different types.

In one embodiment the head portion comprises an upper portion, a body, a lower portion, and two or more apertures. In one embodiment the lower portion couples to the base of the intramedullary structure. In one embodiment the apertures are configured to receive and/or connect to the elongate bodies. In one embodiment the apertures are angled with respect to each other. In various embodiments, the apertures are of the same diameter, different diameters, or combinations thereof. In some embodiments, some or all apertures comprise multiple apertures.

In one embodiment, a bone fixation structure includes a plurality of eleongate bodies, a head portion and an intramedullary portion. In one embodiment, elongate bodies each comprise a proximal end and a distal end. In one embodiment, the head portion includes a plurality of apertures, wherein the apertures are configured to couple to the proximal end of at least one of the elongate bodies. In one embodiment, the intramedullary portion is connected to the head portion and configured to fit within an intramedullary canal. In one embodiment, a first configuration includes a converged position of the elongate bodies, wherein the total distance between the distal ends of the elongate bodies is a first distance. In one embodiment, a second configuration includes a diverged position of the elongate bodies, wherein the total distance between the distal ends of the elongate bodies is a second distance, wherein the second distance is greater than the first distance.

In one embodiment, each of the elongate bodies comprises a longitudinal axis, and the longitudinal axes are not co-planar. In one embodiment, the longitudinal axes of the elongate bodies are not parallel. In one embodiment, the intramedullary portion is an intramedullary nail. In one embodiment, the intramedullary portion is segmented. In one embodiment, the intramedullary portion comprises polyether ether ketone.

In one embodiment, an implantable femoral fixation device, includes a proximal segment, a distal segment and an intermediate segment disposed between the proximal segment and the distal segment. In one embodiment, the implantable femoral fixation device includes a first configuration and a second configuration, the second configuration being larger than the first configuration. In one embodiment, the implantable femoral fixation device includes an anchor. In one embodiment, the anchor comprising a segmented intramedullary structure.

In one embodiment, a method of treating a fracture in a proximal femur, includes the steps of creating an access hole in cortical bone, creating a pathway in cancellous bone through the femoral neck and into the femoral head, creating a cavity in cancellous bone between the cavity and a portion of the inside surface of cortical bone in the femoral head, inserting a femoral fixation device through the access hole, and anchoring the femoral fixation device to an anchor.

In one embodiment, the method includes the step of transforming the femoral fixation device from a radially reduced configuration to a radially expanded configuration. In one embodiment, the method includes providing an anchor, having a proximal end and a distal end, advancing the anchor along a nonlinear path while the anchor is in a flexible state, engaging the bone with the distal end of the anchor, transforming the anchor from the flexible state to a substantially rigid state, and locking the anchor in the substantially rigid state. In one embodiment, the method includes inserting a fixation media to fill at least a portion of the cavity. In one embodiment, the femoral fixation device includes a plurality of elongate bodies, a head portion comprising a plurality of apertures, a first configuration and a second configuration. In one embodiment, the elongate bodies each comprise a proximal end and a distal end. In one embodiment, the apertures are configured to couple to the proximal end of at least one of the elongate bodies. In one embodiment, the head portion is connected to the anchor disposable within an intramedullary canal. In one embodiment, the first configuration includes a converged position of the elongate bodies, wherein the total distance between the distal ends of the elongate bodies is a first distance. In one embodiment, the second configuration includes a diverged position of the elongate bodies, wherein the total distance between the distal ends of the elongate bodies is a second distance, wherein the second distance is greater than the first distance.

In one embodiment, the method includes inserting a plurality of elongate bodies through a plurality of apertures in a head portion of the femoral fixation device. In one embodiment, the elongate bodies each comprise a proximal end and a distal end. In one embodiment, the apertures are configured to couple to the proximal end of at least one of the elongate bodies. In one embodiment, the head portion is connected to the anchor. In one embodiment, the anchor is disposed within an intramedullary canal. In one embodiment, the plurality of elongate bodies comprises a converged region and a diverged region, the converged region of the elongate bodies comprising a first distance between the elongate bodies, the diverged region of the elongate bodies comprising a second distance between the elongate bodies, wherein the second distance is greater than the first distance.

Other features and aspects will become apparent upon reference to the accompanying drawings and description.

### BRIEF DESCRIPTION OF THE DRAWINGS

These and other features, embodiments, and advantages of the present invention will now be described in connection with preferred embodiments of the invention, in reference to the accompanying drawings. The illustrated embodiments, however, are merely examples and are not intended to limit the invention.

FIG. 1 is a schematic partial cross-sectional front view of a femur.

FIG. 2 is a schematic partial cross-sectional front view of a standard femoral head fixation screw with a plate.

FIG. 3 is a schematic partial cross-sectional front view of the standard femoral head fixation device in FIG. 2.

FIG. 4 is a schematic partial cross-sectional front view of a proximal femur fixation device with a segmented intramedullary device anchor according to an embodiment of the present invention.

FIG. 5 is a schematic partial cross-sectional front view of an injectable mechanical composite implant according to an embodiment of the present invention.

FIG. 6 is a schematic partial cross-sectional front view of a radial rod bone cement device in a reduced configuration according to an embodiment of the present invention.

FIG. 6A is a schematic partial cross-sectional side view of the radial rod bone cement device of FIG. 6.

FIG. 6B is a schematic partial cross-sectional side view of the radial rod bone cement device of FIG. 6.

FIG. 7 is a schematic partial cross-sectional front view of the radial rod bone cement device of FIG. 6 in an expanded configuration.

FIG. 7A is a schematic partial cross-sectional side view of the radial rod bone cement device of FIG. 6 in an expanded configuration.

FIG. 8 is a schematic partial cross-sectional front view of an expandable intertrochanteric frame device in a reduced configuration according to an embodiment of the present invention.

FIG. 9 is a schematic partial cross-sectional front view of the expandable intertrochanteric frame device of FIG. 8 in an expanded configuration.

FIG. 10 is a schematic partial cross-sectional front view of a shape memory cortical bone support device in a first configuration according to an embodiment of the present invention.

FIG. 10A is a schematic front view of the shape memory cortical bone support device of FIG. 10.

FIG. 11 is a schematic partial cross-sectional front view of the shape memory cortical bone support device of FIG. 10 in a second configuration.

FIG. 11A is a schematic front view of the shape memory cortical bone support device of FIG. 11.

FIG. 12 is a schematic partial cross-sectional front view of an expandable tube device in a reduced configuration according to an embodiment of the present invention.

FIG. 12A is a schematic partial cross-sectional side view of the expandable tube device of FIG. 12.

FIG. 12B is a close up of the schematic partial cross-sectional side view of the expandable tube device of FIG. 12A.

FIG. 13 is a schematic partial cross-sectional front view of the expandable tube device of FIG. 12 in an expanded configuration.

FIG. 13A is a schematic partial cross-sectional side view of the expandable tube device of FIG. 13.

FIG. 14A is a schematic front view of a beveled bellows structure in a radially reduced configuration according to an embodiment of the present invention.

FIG. 14B is a schematic front view of the beveled bellows structure of FIG. 14A in a radially expanded configuration.

FIG. 15A is a schematic partial cross-sectional front view of a bevel structure in a radially reduced configuration according to an embodiment of the present invention.

FIG. 15B is a schematic partial cross-sectional front view of the bevel structure of FIG. 15A in a radially expanded configuration.

FIG. 16 is a schematic partial cross-sectional front view of a beveled structure in a radially expanded configuration according to an embodiment of the present invention.

FIG. 17 is a schematic partial cross-sectional front view of a cortical support structure according to an embodiment of the present invention.

FIG. 18A is a schematic partial cross-sectional side view of an expandable cortical support structure in a reduced configuration according to an embodiment of the present invention.

FIG. 18B is a schematic partial cross-sectional side view of the expandable cortical support structure of FIG. 18A in an expanded configuration.

FIG. 19 is a schematic partial cross-sectional side view of a cortical support structure according to an embodiment of the present invention.

FIG. 20 is a schematic partial cross-sectional front view of a directionally actuatable cortical support structure in a first configuration according to an embodiment of the present invention.

FIG. 20A is a schematic close up front view of the directionally actuatable cortical support structure of FIG. 20.

FIG. 21 is a schematic partial cross-sectional front view of the directionally actuatable cortical support structure of FIG. 20 in a second configuration.

FIG. 21A is a schematic close up front view of the directionally actuatable cortical support structure of FIG. 21.

FIG. 22 is a schematic partial cross-sectional front view of an articulatable material delivery device according to an embodiment of the present invention.

FIG. 23 is a schematic partial cross-sectional front view of the articulatable material delivery device of FIG. 22.

FIG. 24 is a schematic partial cross-sectional front view of an anchored cable tensioning device according to an embodiment of the present invention.

FIG. 25 is a schematic partial cross-sectional front view of multiple anchored cable tensioning devices according to an embodiment of the present invention.

FIG. 26 is a schematic front view of an anchored cable tensioning device according to an embodiment of the present invention.

FIG. 27 is a schematic partial cross-sectional front view of an anchored cable tensioning device and a segmented intramedullary structure according to an embodiment of the present invention.

FIG. 28 is a schematic perspective view of an embodiment of a femoral fixation device.

FIG. 29 is a schematic front view of the embodiment of FIG. 28.

FIG. 30 is a schematic perspective view of an embodiment of an intramedullary structure.

FIG. 31 is a schematic perspective view of an embodiment of an elongate body.

FIG. 32 is a schematic perspective view of an embodiment of a head portion.

FIG. 33 is a right side view of the embodiment of FIG. 32.

FIG. 34 is a rear view of the embodiment of FIG. 32.

FIG. 35 is a bottom view of the embodiment of FIG. 32.

FIG. 36 is a front view of another embodiment of a head portion.

FIG. 37 is a b of the embodiment of FIG. 36.

FIG. 38 is a right side view of the embodiment of FIG. 36.

FIG. 39 is a schematic perspective view of another embodiment of a head portion.

FIG. 40 is a rear view of the embodiment of FIG. 39.

FIG. 41 is a front view of the embodiment of FIG. 39.

FIG. 42 is a schematic perspective view of an embodiment of a cross-screw distal end of an intramedullary structure.

FIG. 43 is a schematic perspective view of an embodiment of a radially-expandable distal end of an intramedullary structure.

FIG. 44 is a schematic perspective view of an embodiment of a polymer distal end of an intramedullary structure.

Throughout the figures, the same reference numerals and characters, unless otherwise stated, are used to denote like features, elements, components or portions of the illustrated embodiments. In certain instances, similar names may be used to describe similar components with different reference numerals which have certain common or similar features. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION

In accordance with the present disclosure, various embodiments of a bone fixation apparatus, systems and methods are provided. Various embodiments are directed to a proximal femur fixation apparatus, systems and methods. In one embodiment a fixation apparatus comprises an intramedullary structure and one or more elongate bodies extending from the intramedullary structure in to the femoral neck or femoral head.

Various embodiments of a proximal femur fixation apparatus as disclosed herein allow the surgeon to compress the fracture site after placing the fixation apparatus in the bone and fixating the fractured or damaged bone segments. Fractures or damaged sections of bone can be fixated and compressed together to assist in the healing of the bone. In some embodiments bone segment or fracture compression may be expressed in terms of compressive force applied to bring bone segments together with a device. In some embodiments compression may be expressed in terms of the tensile force applied to a tensioning mechanism to bring bone segments together with a device. In some embodiments, compression can be described in terms of a distance, such as the distance that bone segments are brought together in compression. In one embodiment compression is expressed in terms of the decrease in the decrease in axial length of the device along the direction of the compression. In one embodiment the distance associated with compression is proportional to the amount of compressive or tensile force applied to the device. In one embodiment a proximal femur fixation structure can be configured to provide substantially one level or one distance in compression. In one embodiment a proximal femur fixation structure can be configured to provide varying levels or ranges of compression. In one embodiment a proximal femur fixation structure can provide a smooth, continuous transition between levels of compression. In one embodiment a proximal femur fixation structure can provide a discrete transition between levels of compression. In one embodiment a proximal femur fixation structure can provide a discrete transition between levels of compression with a ratcheting action.

In one embodiment a proximal femur fixation structure can provide no compression. In various embodiments a proximal femur fixation structure can be configured to provide a single compression distance with a value in the range of about 1 mm to 5 mm. In various embodiments a proximal femur fixation structure can be configured to provide 1 mm, 2 mm, 3 mm, 4 mm, or 5 mm of compression. In one embodiment a proximal femur fixation structure is configured to provide anywhere in the range of about 1 mm to 5 mm of compression. Proper compression of the fracture site after the fracture has been reduced and proximal and distal fixation is in place helps ensure that the reaches full reduction at the fracture site. In one embodiment, an additional benefit of compression is that it takes some of the load off the implant which will help in implant longevity.

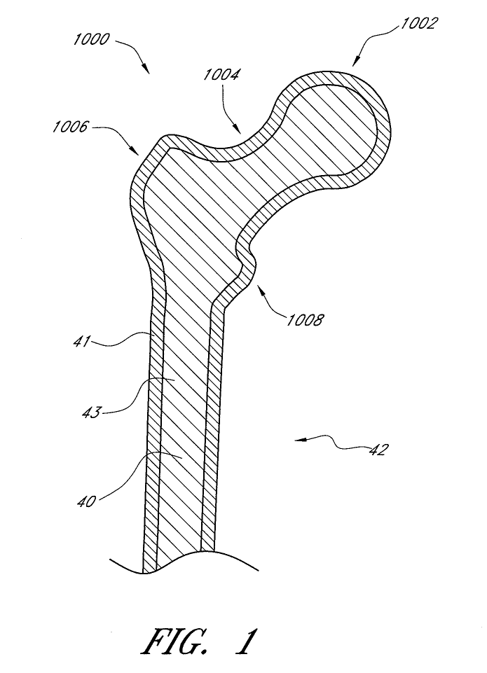

Various embodiments of a proximal femur fixation structure as disclosed herein may list various parameters, such as sizes, lengths, diameters, widths, curvatures and geometry that can conform to or be implanted based on various parameters of bones and of structures in which embodiments of the devices may be configured to be implanted. Listings provide some examples, but should not be read to limit the disclosure to those specific dimensions or characteristics. For example, dimensions of a device and its various size and shape and feature characteristics can vary depending on parameters of the bone and/or patient, the type of fracture, and other factors. Embodiments of proximal femur fixation structures are scalable. A schematic illustration of a proximal portion of a femur 1000 is provided at FIG. 1. The femur 1000 is a long bone 42 with an intramedullary canal 40. The bone 42 has a hard outer bone cortex comprised of cortical bone 41 and a softer, more porous cancellous bone 43 in the interior of the bone 42. Generally, cortical bone 41 is stronger than cancellous bone 43. Osteoporosis can result in the degeneration of, and weakening of the cancellous bone 43. A variety of conditions can lead to the reduction of structural integrity of cancellous bone 43 and/or cortical bone 41. For example, in some instances age, calcium deficiency, disease, or other conditions can lead to cancellous bone 43 losing structural integrity. In some cases, cancellous bone 43 can weaken and can have a consistency like putty. In some cases this can be quite pronounced, with older patients or osteoporotic cancellous bone 42 having the consistency of putty that can be scooped out with relative ease. In some instances structural bone loss can occur in cancellous bone 43.

The proximal end of the femur 1000 has a femoral head 1002, a femoral neck 1004, a greater trochanter 1006 and a lesser trochanter 1008. Fractures 44 can occur anywhere in the femur 1000. One type of common fracture in the proximal femur 1000 includes fractures across or along the length of the femur 1000 and the intramedullary canal 40. Another type of common fracture in the proximal femur 1000 includes fractures across or along the length of the neck 1004 or around the femoral head 1002, greater trochanter 1006 and/or lesser trochanter 1008. This second type of proximal femoral fracture is often associated with falls and in some instances may be called hip fractures.

There is a significant unmet clinical need for better devices and methods for treatment of proximal femoral fractures. Conventional technologies include various uses of screws, cannulated screws, compression hip screws, plates in trochanteric or interochanteric implants for treating femoral head and/or intertrochanteric fractures. Among patients treated with conventional technologies, studies have found that only 40% regain their pre-operative mobility, and only 24% regain the pre-operative function. The risk of these types of fractures increases in osteoporotic bone. As populations get older the incidence of these types of hip fractures will continue to increase.

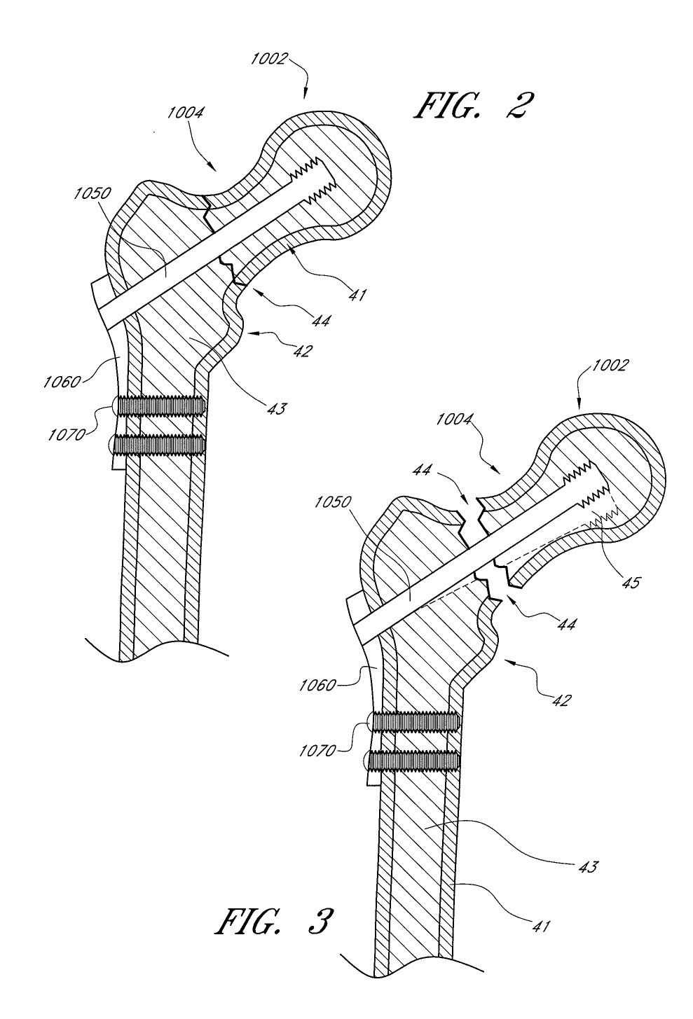

One problem associated with soft, osteoporotic bone, as is commonly observed in the elderly, is a loss of mobility due to anatomic changes in the femoral head 1002 location. Conventional treatment of proximal femoral fractures with conventional devices can often lead to the use of hard metal structures such as nails or screws placed inside weak, osteoporotic bone. For example, one schematic example of a conventional treatment of a proximal femoral fracture is illustrated in FIGS. 2 and 3. Some conventional applications involve inserting a rigid screw 1050 to span the inside of the fracture 44, seating the screw 1050 in cancellous bone 43 and anchoring the device in the more rigid cortical bone 41 with a plate 1060 and screws 1070. Even if an initial fusion or treatment is successful in fusing some or most of the bone segments after a fracture, over time, conventional implants may start to migrate with respect to the femoral head 1002. In certain instances, an implant might stay in place with an anchor mechanism while the surrounding soft, cancellous 43 or osteoporotic bone inside or near the femoral head 1002 is unable to support the implant. Convention implants can carve out a cavity 45 or a path inside the soft or osteoporotic bone 43 that leads to migration of implant, the femoral head 1002, or both. Conventional attempts to fix this problem with the addition of supplemental screws or other plates and structures tend to increase damage in the bone 42, resulting in more bone loss and more significant potential for migration and injury. Fractures 44 can reopen, or new fractures 44 can occur as the conventional implant migrates within the bone 42.

In various embodiments a segmented intramedullary structure 300 can be used in a femur 1100. In various embodiments, the size of access holes, parts, and angles of approach or other characteristics are configured for use with a femur 1000. In one embodiment a segmented intramedullary structure 300 can be used to bridge a fracture 44. In one embodiment a segmented intramedullary structure 300 can be used as an anchor to support a device to bridge a fracture 44.

In some embodiments the segmented intramedullary structure 300 can be used to bridge a fracture 44, as discussed in any of the various long bone 42 applications and embodiments discussed above. In one embodiment a segmented intramedullary structure 300 can be used to bridge a fracture 44 in or near the femoral neck 1004. In one embodiment a segmented intramedullary structure 300 is used to bridge a fracture 44 along the intramedullary canal 40. For example, in one embodiment a segmented intramedullary structure 300 can be inserted through an access hole 46 in the greater trochanter 1006. In various embodiments a segmented intramedullary structure 300 can be inserted through an access hole 46 proximate to the greater trochanter 1006. In various embodiments a segmented intramedullary structure 300 can be inserted in a retrograde approach proximate the knee, antegrade approach, or other approach. In one embodiment, the steps described in extracting or removing a segmented intramedullary structure 300 may be applied to a femur 1000. In one method of assembly, manufacture, or construction of the segmented intramedullary structure 300, a surgeon could assemble a modular or custom segmented intramedullary structure 300 while in the operating room for use in a femur 1000.

Various embodiments of the segmented intramedullary structure 300 can be used to anchor an attachment. In one embodiment the attachment connects to the segmented intramedullary structure 300 at an interface. The interface 1150 on the segmented intramedullary structure 300 corresponds to an interface 1105 on the femoral fixation device 1100. In one embodiment the interface 1155 of the segmented intramedullary structure 300 threadably engages the interface 1105 of the femoral fixation device 1100. In one embodiment the segmented intramedullary structure 300 and femoral fixation device 1100 interface is configured to prevent rotation or relative motion between the segmented intramedullary structure 300 and the femoral fixation device 1100. In one embodiment the segmented intramedullary structure 300 interface 1155 locks to the femoral fixation device 1100 interface 1105. In one embodiment the segmented intramedullary structure 300 interface 1155 is similar to a throughbore 112. In various embodiments, the attachment can be a separate device. In one embodiment the attachment is a femoral fixation device 1100.

In various embodiments a femoral fixation device 1100 is configured to treat a proximal femoral fracture 44 by bridging the interior of the fracture through a femoral neck 1004 and/or a femoral head 1002. In various embodiments the femoral fixation device 1100 has a proximal segment 1102, an intermediate segment 1104, and a distal segment 1106.

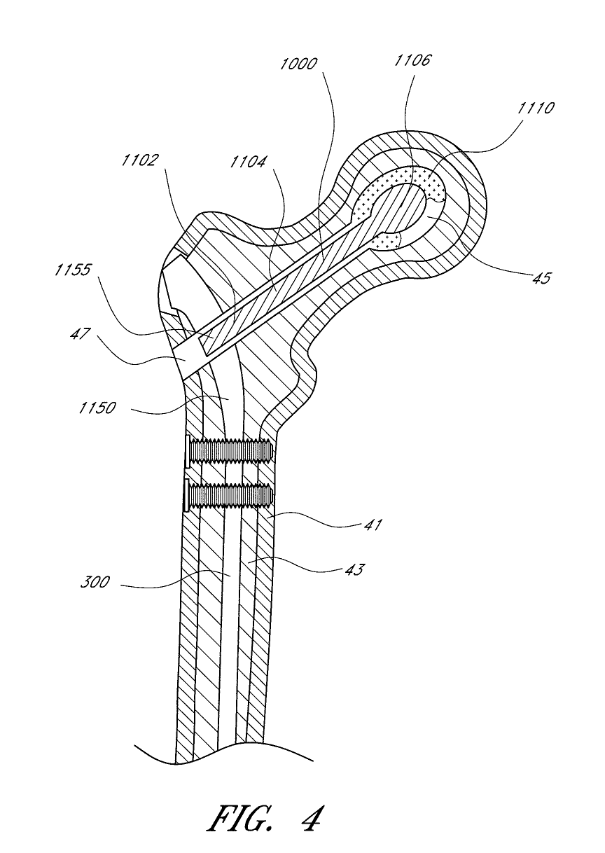

In various embodiments the femoral fixation device 1100 is configured to attach to an anchor 1150. In one embodiment the proximal segment 1102 is removably attachable to an anchor 1150. In various embodiments the anchor 1150 can be a bone screw 1070, a plate 1060, or some other attachment to cortical bone 41. In one embodiment the anchor 1150 is a segmented intramedullary structure 300. FIG. 4 illustrates a proximal femur fixation device with a segmented intramedullary device anchor according to an embodiment of the present invention. For the purposes of illustration, many of the figures include a segmented intramedullary structure 300 as an anchor 1150 for the femoral fixation device 1100. However, the embodiments of femoral fixation devices 1100 can be used with or without any other type of anchor 1150. In various embodiments, the femoral fixation device 1100 is configured for implantation and removal or extraction

In some embodiments the femoral fixation device 1100 may have a cavity 45 in the cancellous bone 43 around all or a portion of the femoral fixation device 1100. In one embodiment cancellous bone 43 may be displaced by a femoral fixation device 1100. In one embodiment some or substantially all of the cancellous bone 43 in the femoral head 1002 and/or femoral neck 1004 may be extracted before, during or after insertion of a femoral fixation device 1100. In one embodiment cancellous bone 43 is removed with a drill, bore, or reaming device. In one embodiment a cavity 45 is formed and shaped to support insertion and to reduce potential motion of a femoral fixation device 1100. In one embodiment a cavity 45 is formed to remove substantially all cancellous bone 43 between a femoral fixation device 1100 and cortical bone 41. In one embodiment forming a cavity 45 involves reaming the cavity 45. In certain cases cancellous bone 43 may be weakened due to osteoporosis, damage, or other conditions and is removed in order to create a more stable environment for fixation.

Any type of fixation media 1110, such as bone cement, filler, or any type of material may be inserted into a cavity 45. In various embodiments the fixation media 1110 can be a polymer, absorbable, biocompatible, composite, thrombogenic material, thrombo-resistant material, bone growth material, pharmaceutical, drug, drug eluting, antibiotic, growth factor, surgical fluid, and/or other material. In one embodiment the fixation media 1110 stimulates natural bone formation. In one embodiment the fixation media 1110 is absorbed at the same or similar rate as natural bone formation. In one embodiment the fixation media 1110 has a fluid state and hardens into a more solid state. In various embodiments the fixation media 1110 is a polymer bone cement such as PMMA (polymethyl methacrylate), calcium phosphate cement, a bone graft substitute, a collagen matrix colloid, or any other material that provides sufficient strength upon hardening. In various embodiments the fixation media 1110 may be a non-absorbable PMMA product, such as Surgical Simplex P, Palacose, Zimmer Regular, Zimmer Low Viscosity (LVC), CMW-1, CMW-3, Osteopal, Osteobond, Endurance bone cement, or a similar product. Various embodiments of the fixation media 1110 may be a non-absorbable PMMA product with antibiotics, such as Palacos R with gentamycin, Surgical Simplex P with tobramycin, or a similar product. In various embodiments the fixation media 1110 may be an absorbable product, such as Norian SRS, calcium phosphate cement (CPC), calcium phosphate hydraulic cement (CPHC), sodium citrate modified calcium phosphate cement, hydroxyapatite (HA) cement, hydroxyapatite calcium phosphate cements (CPCs); a beta-TCP-MCPM-CSH cement [beta-tricalcium phosphate (beta-TCP), monocalcium phosphate monohydrate (MCPM), and calcium sulfate hemihydrate (CSH)]; a bioactive bone cement (GBC) with bioactive MgO—CaO—SiO2-P2O5-Caf2 glass beads and high-molecular-weight polymethyl methacrylate (hPMMA); a tricalcium phosphate (TCP), tetracalcium phosphate (TTCP), and dicalcium phosphate dehydrate (DCPD) bone cement with dense TCP granules; an hPMMA with delta- or alpha-alumina powder (delta-APC or alpha-APC); a similar product; or any other material that provides sufficient strength upon hardening.

In various embodiments the fixation media 1110 can be thermo-mechanically or thermo-chemically activated. In one embodiment the fixation media 1110 comprises a thermo-chemically activated material which has physical properties that change between a first and second state. For example, the material may be flexible and deformable at a first state and harder and more rigid at a second state. This can be accomplished by changing factors such as the molecular structure of chemical components of the fixation media 1110 from one state to another. In one embodiment the fixation media 1110 comprises thermo-chemically activated materials which have physical properties which may change between a first state and second state by chemical, thermal, or other processes which change the molecular structure of a material, and thus the physical properties of the material. Embodiments of these processes may include, but are not limited to: changing the temperature of the material, exposing the material to gamma radiation and altering the crosslinking bonds between molecular chains in the material, exposing the material to ultraviolet radiation causing the material to cure and harden, exposing the material to a second material allowing cross-linking and molecular bonding, allowing the material to harden over time by increasing the crystallinity within the molecular structure, and other methods that alter the bonding between the molecules in the material and correspondingly alter its material properties. In one embodiment thermo-chemically activated materials may also be referred to as thermoplastic. In various embodiments the fixation media 1110 may comprise a thermoplastic biocompatible polymer or polymer blend comprising polymers such as polylactic acid (PLA), poly ε-caprolactone (PCL), trimethylene carbonate (TMC), polyglycolic acid (PGA), poly l-lactic acid (PLLA), poly d-l-lactide (PDLLA), poly-D,L-lactic acid-polyethyleneglycol (PLA-PEG) or other biocompatible polymers. Each of these polymers has a glass transition temperature Tg such that when raised to a temperature above its Tg, the polymer is rubbery, flexible and substantially deformable. When lowered to a temperature below its Tg, the polymer is crystallized and substantially hardened. Each of these polymers or blends is capable of being transformed by the application of energy to a first thermo-chemical state, in which it is at a temperature above its glass transition temperature Tg. When, through dissipation of energy, the temperature is reduced to below Tg, the polymer or blend is at a second thermo-chemical state. These thermoplastic properties of the polymers allow them to be repetitively heated to above Tg, and subsequently cooled to below Tg, moving repeatedly between the first and second thermo-chemical states. In one embodiment a fixation media 1110 is any polymer having a glass transition temperature Tg that is above body temperature, but below the temperature known to cause thermal necrosis of tissues. In one embodiment a fixation media 1110 has a blend that is crystallized and substantially rigid at human body temperature, and has a Tg which ranges from about 10° C. above body temperature to about 35° C. above body temperature. This acceptable Tg range is between about 50° C. and about 80° C., and preferably between about 55° and about 65° C. In one embodiment a fixation media 1110 comprises a blend of polymers such as PCL and PLA, or PCL and PGA.

In various embodiments the fixation media 1110 comprises one or more biocompatible polymers, aliphatic polyesters, polyglycolide, poly(dl-lactide), poly(l-lactide), poly(δ-valerolactone), polyhydroxybutyrate; polyanhydrides including poly[bis(p-carboxyphenoxy) propane anhydride], poly(carboxy phenoxyacetic acid), poly(carboxy pheoxyvaleric acid); polyphosphazenes including aryloxyphosphazene polymer and amino acid esters; poly (ortho esters); poly(p-dioxane); poly(amino acids) including poly(glutamic acid-co-glutamate); erodable hydrogels; and natural polymers including collagen (protein) and chitosan (polysaccharide).

In various embodiments the fixation media 1110 may further include at least one bioactive material to promote growth of bone material and accelerate healing of fractures. These bioactive materials include but are not limited to hydroxylapatite, tetracalcium phosphate, β-tricalcium phosphate, fluorapatite, magnesium whitlockite, β-whitlockite, apatite/wollastonite glass ceramic, calcium phosphate particle reinforced polyethylene, bioactive glasses, bioactive glass ceramics, polycrystalline glass ceramics, and polyethylene hydroxylapatite.

In one embodiment the entire cavity 45 is filled with fixation media 1110. In one embodiment at least part of a cavity 45 is filled with fixation media 1110. In one embodiment the fixation media 1110 is inserted in the cavity 45 prior to femoral fixation device 1100 insertion. In one embodiment the fixation media 1110 is inserted in the cavity 45 during femoral fixation device 1100 insertion. In one embodiment the fixation media 1110 is inserted in the cavity 45 after femoral fixation device 1100 insertion. In one embodiment the fixation media 1110 is inserted with a cement insertion device. In one embodiment the fixation media 1110 is inserted via one or more holes or channels in a femoral fixation device 1100.

In one embodiment the femoral fixation device 1100 is inserted through an access hole 47 in the cortical bone 41. In one embodiment the access hole 47 for femoral fixation device 1100 insertion is separate from an access hole 47 for insertion of a segmented intramedullary structure 300. In one embodiment the access hole 47 for femoral fixation device 1100 insertion is the same as the access hole 47 for insertion of a segmented intramedullary structure 300. In one embodiment tissue, such as skin, muscle, tendons, fat is parted to access the access hole or holes 46 and 47. In one embodiment a single incision in tissue is used to access the access holes 46 and 47. In one embodiment a tissue incision can be moved or translated along the cut tissue to move between accessing one access hole 46 and another access hole 47. In one embodiment a single incision in tissue is used to access each of the access holes 46 and 47. In one embodiment an incision in tissue is a lateral incision.

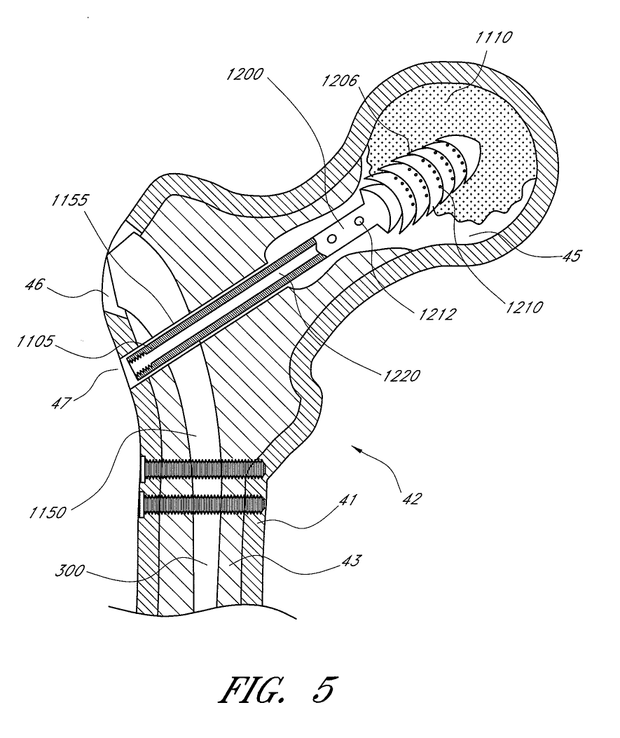

FIG. 5 illustrates an injectable mechanical composite implant 1200 according to an embodiment of a femoral fixation device 1100. In one embodiment the injectable mechanical composite implant 1200 compresses the head 1002 with lag screw drawing. In one embodiment the injectable mechanical composite implant 1200 compresses the head 1002 with cable tensioning. In one embodiment the injectable mechanical composite implant 1200 comprises a cannulated lag screw configured for fixation media 1110 injection. In one embodiment the injectable mechanical composite implant 1200 comprises a cannulated compression screw configured for fixation media 1110 injection. In one embodiment the injectable mechanical composite implant 1200 comprises a lumen for fixation media 1110 injection.

In one embodiment the injectable mechanical composite implant 1200 has a proximal segment 1202, an intermediate segment 1204, and a distal segment 1206. In one embodiment the distal segment 1206 is configured to interlock with hardened fixation media 1110. In one embodiment the distal segment 1206 comprises threads. In one embodiment the distal segment 1206 can be broken off from the intermediate segment 1204, with the intermediate segment 1204 and the proximal segment 1202 removable from the bone 42. In one embodiment the distal segment 1206 comprises one or more ports 1210 for bone cement 1100 injection via the lumen 1220. In one embodiment the intermediate segment 1204 comprises one or more ports 1212 for bone cement 1100 injection via the lumen 1220. In one embodiment the injectable mechanical composite implant 1200 is inserted through an anchor 1150 and across a fracture site 44 in a radially reduced configuration and then expanded.

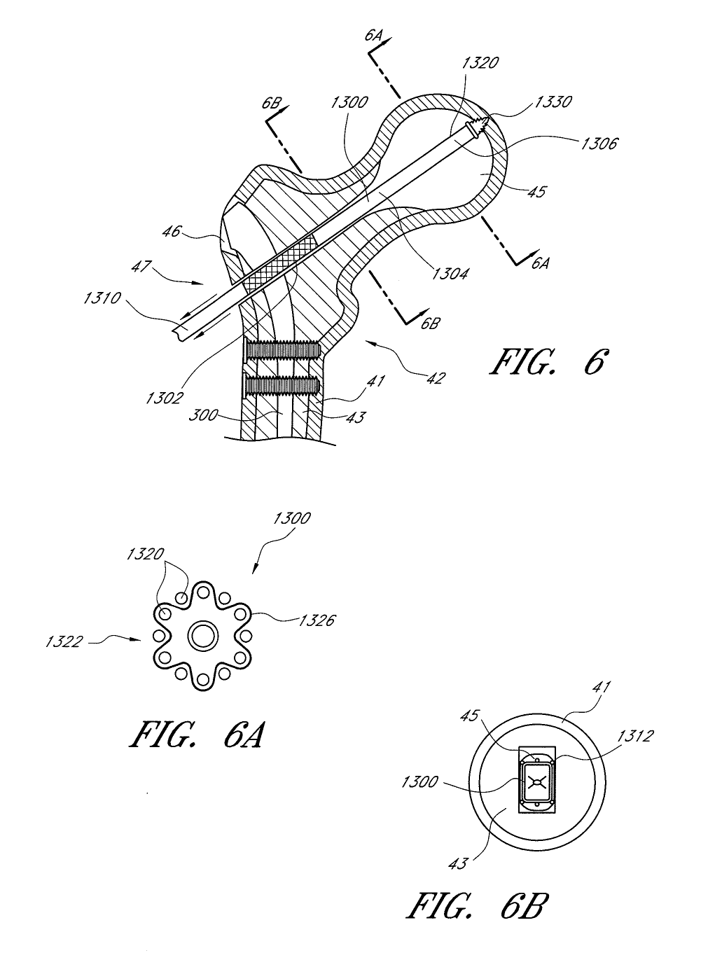

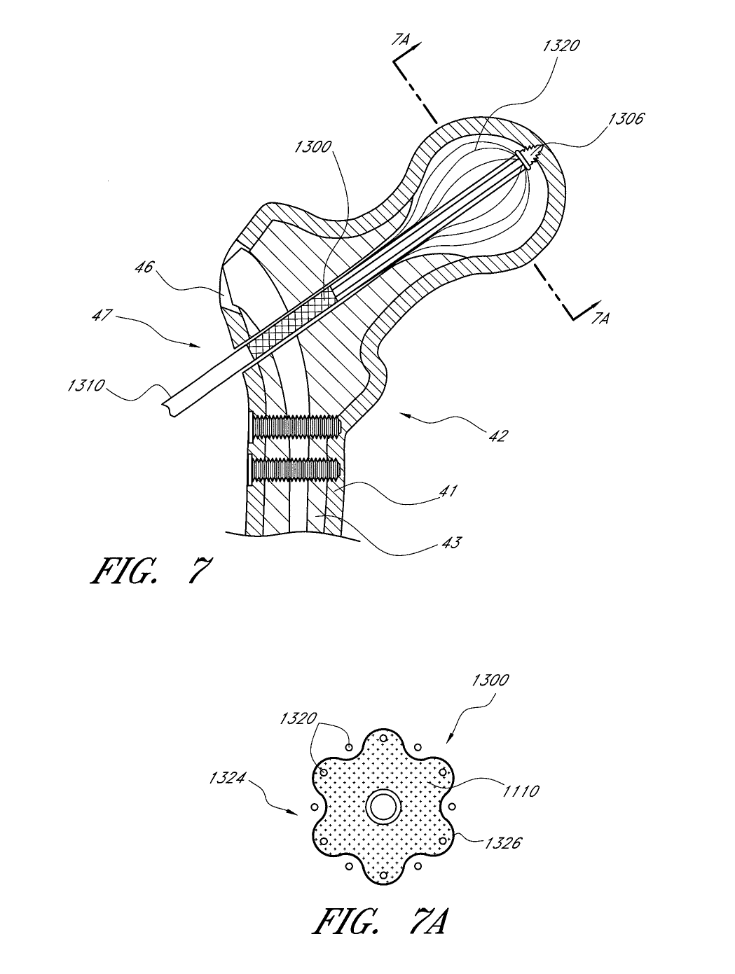

In one embodiment of a femoral fixation device 1100, an expandable radial bone support device 1300 comprises a proximal segment 1302, an intermediate segment 1304, and a distal segment 1306, a tensioning element 1310 and a plurality of rods 1320. FIGS. 6-7A illustrate an expandable radial bone support device according to an embodiment of the present invention. In one embodiment, two adjacent holes are drilled into the femur 1000, as is represented by the dotted circles with reference numbers 1312, then the cavity 45 is broached into a non-circular cross-section (such as a rectangle shape) to prevent rotation of the femoral fixation device 1100 with respect to the fracture 44 within the bone 42. In one embodiment the intermediate segment 1304 has a corresponding non-circular cross sectional shape to prevent rotation within the cavity 45. In one embodiment the distal segment 1306 of the expandable radial bone support device 1300 has a threaded tip 1330 configured to bite in to and anchor the distal segment 1306 in cortex in the cortical bone 41. The threaded tip 1330 is advanced in to cortical bone 41.

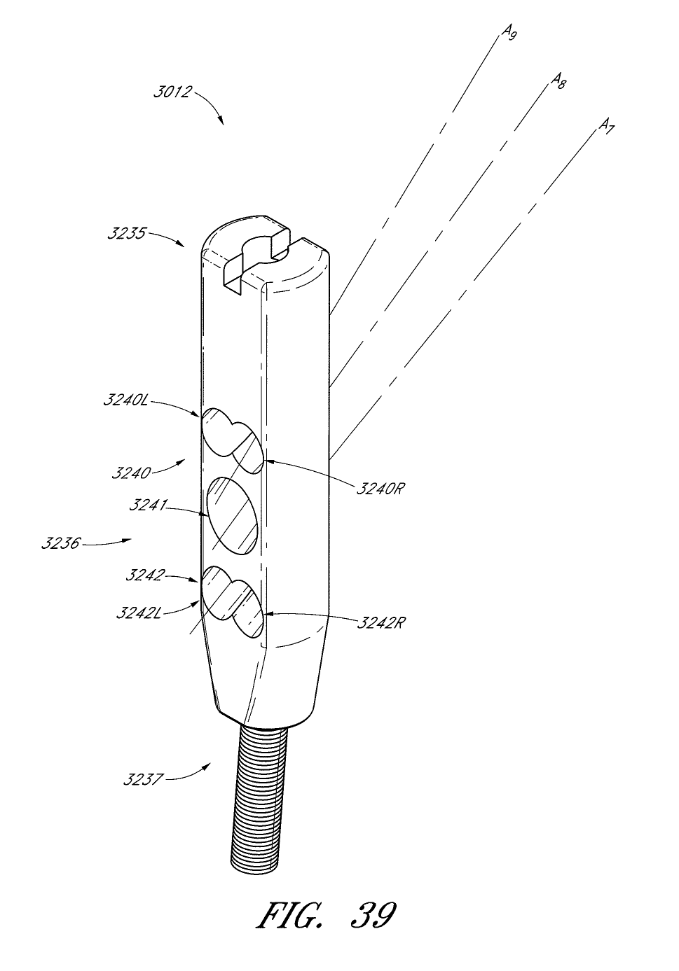

In one embodiment the plurality of rods 1320 is configured to expand radially away from the longitudinal axis of the expandable radial bone support device 1300 between a radially reduced configuration 1322 and a radially expanded configuration 1324. In one embodiment the plurality of rods 1320 expands in a manner similar to an umbrella. In one embodiment the plurality of rods 1320 expands mechanically in to a radially expanded configuration 1324 that appears similar to an egg beater. In various configurations, the radial cross sectional view of the radially reduced configuration 1322 and/or the radially expanded configuration 1324 can be circular, oval, elliptical, rectangular, square, triangular, or some other shape. In one embodiment proximal movement of a tensioning element 1310 moves the plurality of rods 1320 from the radially reduced configuration 1322 to the radially expanded configuration 1324. In one embodiment the tensioning member 1310 is similar to an elongate member 350. In one embodiment the tensioning member 1310 is a cable. In one embodiment the tensioning member 1310 is locked at the anchor 1150. In one embodiment the tensioning member 1310 is lockable with a two-part distal collet assembly 132 as described above with respect to FIG. 38 of U.S. Provisional No. 61/150,134, filed Feb. 5, 2009, which is incorporated by reference in its entirety herein. In one embodiment the tensioning member 1310 is lockable with a cable collet anchor 272 arrangement as described above with respect to FIG. 68 of U.S. Provisional No. 61/150,134, filed Feb. 5, 2009. In one embodiment a cable tensioner assembly 200 is used to tension the tensioning element 1310.

In one embodiment fixation media 1110 fills the cavity 45 in and around the expandable radial bone support device 1300. In one embodiment the fixation media 1110 is advanced in to the cavity 45 while maintaining tension in the tensioning member 1310. In one embodiment a membrane 1326 is disposed along the length of the expandable radial bone support device 1300. The membrane 1326 is configured to fill with fixation media 1110. In one embodiment the outside of the membrane 1326 is filled with fixation media 1110. In one embodiment the membrane 1326 is a mesh with an open structure allowing fixation media 1110 and tissue ingrowth across the membrane 1326.

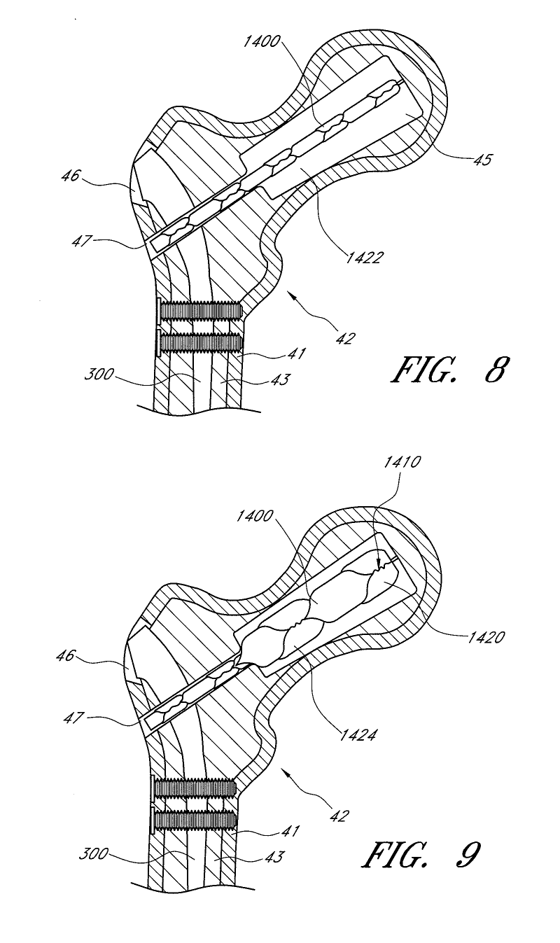

In one embodiment of a femoral fixation device 1100, an expandable intertrochanteric frame device 1400 comprises a frame 1410 and an inflatable membrane 1420. FIG. 8 illustrates an expandable intertrochanteric frame device 1400 in a reduced configuration 1422 according to an embodiment of the present invention. FIG. 9 illustrates the expandable intertrochanteric frame device 1400 in an expanded configuration 1424. In one embodiment the inflatable membrane 1420 is configured to fill with fixation media 1110. In one embodiment the expandable intertrochanteric frame device 1400 is configured to be surrounded with fixation media 1110.

In one embodiment the frame 1410 is a web-like structure. In one embodiment the frame 1410 is a stent. In various embodiments the frame 1410 can have patterns that are adaptable to a variety of lengths, diameters, density of repeatable patterns, wire thicknesses, web areas, and other structural characteristics such that the general frame 1410 shape can be configured to a particular bone morphology and size. The frame 1410 may also be configured with more than one pattern along its length or diameter if needed to better conform to the desired geometry. The frame 1410 may have a unique configuration which is constructed from wire, woven, machined, laser cut, or chemically etched.

In one embodiment the shape of the expandable intertrochanteric frame device 1400 can be configured to conform to the shape of the cavity 45. In one embodiment substantially all cancellous bone 43 is removed from the femoral head 1002 and/or femoral neck 1004. In various embodiments the shape of the expandable intertrochanteric frame device 1400 can remain relative constant or vary along the length or width or depth of the expandable intertrochanteric frame device 1400. In one embodiment the shape of the expandable intertrochanteric frame device 1400 is configured to correspond to the shape of the interior of the cortical bone 41 of the femoral head 1002.

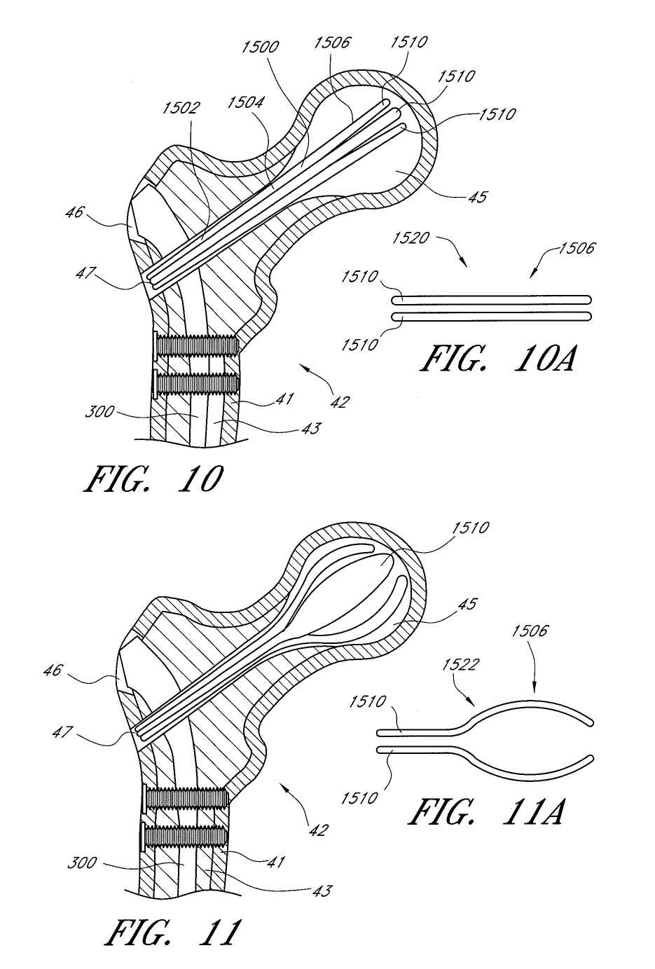

In one embodiment of a femoral fixation device 1100, a shape memory cortical bone support device 1500 comprises one or more elongate bodies 1510. The elongate body 1510 comprises a proximal segment 1502, an intermediate segment 1504, and a distal segment 1506. The elongate body 1510 has a first, straightened configuration 1520 for insertion or removal in to the bone 42, and a second, deflected configuration 1522 for bracing cortical bone 41. In one embodiment the distal segment 1506 is substantially straight in the straightened configuration 1520 and is curved in the deflected configuration 1522. In one embodiment the proximal segment 1502 is removably attachable to an anchor 1105. FIGS. 10-10A illustrate a shape memory cortical bone support device 1500 in a first, straightened configuration 1520 according to an embodiment of the present invention. FIGS. 11-11A illustrate the shape memory cortical bone support device 1500 in a second, deflected configuration 1522.

In one embodiment the elongate body 1510 comprises a shape memory material. In one embodiment the elongate body 1510 comprises Nitinol. In one embodiment the elongate body 1510 changes shape depending on material temperature. In one embodiment the elongate body 1510 has a first, straightened configuration 1520 at a temperature below body temperature, and a second, deflected configuration 1522 at or near body temperature. In one embodiment the deflected configuration 1522 of the elongate body 1510 curves in one dimension. In one embodiment the deflected configuration 1522 of the elongate body 1510 curves in two dimensions. In one embodiment the deflected configuration 1522 of the elongate body 1510 curves in three dimensions. In one embodiment the deflected configuration 1522 of the elongate body 1510 is shaped like a spoon.

In one embodiment of a shape memory cortical bone support device 1500, a single elongate body 1510 is configured with a deflected configuration 1522 oriented to conform to a cephalad inner surface of the cortical bone 41 of the femoral head 1002. In one embodiment a single elongate body 1510 is configured with a deflected configuration 1522 oriented to conform to a caudal inner surface of the cortical bone 41 of the femoral neck 1004. In various embodiments of a shape memory cortical bone support device 1500, two, three, four or more elongate bodies 1510 are configured with respective deflected configurations 1522 to conform to a portion of the inner surface of the cortical bone 41 of the femoral head 1002 and/or femoral neck 1004. In various embodiments, multiple elongate bodies 1510 are configured to cover roughly 360 degrees around the circumference of the inner surface of the cortical bone 41 in a femoral head 1002 and/or femoral neck 1004. For example, in one embodiment four elongate bodies can cover roughly 90 degrees each, and are coupled to cover a total circumferential area of roughly 360 degrees.

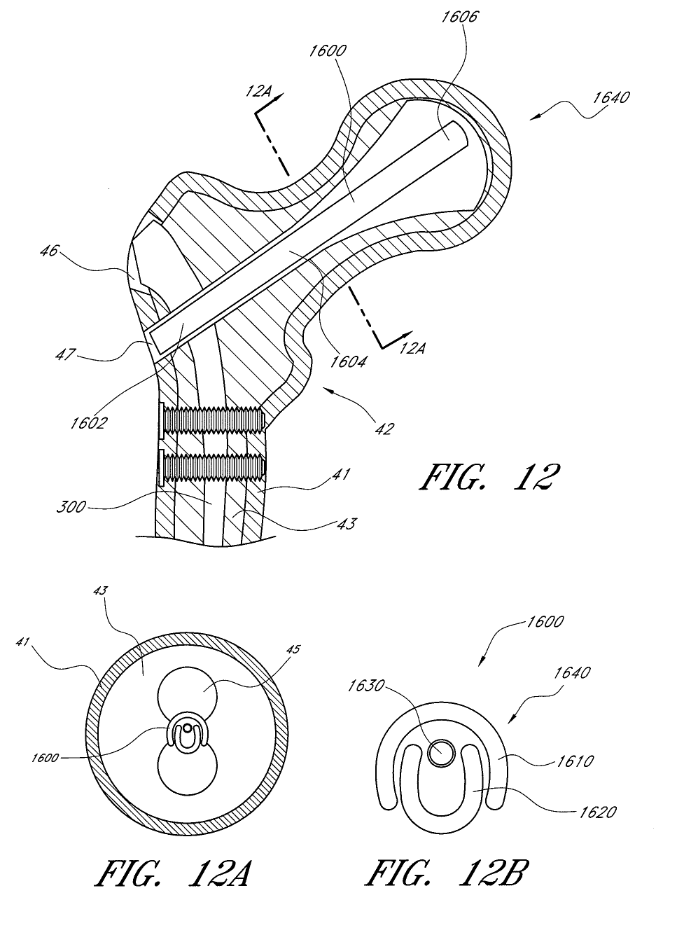

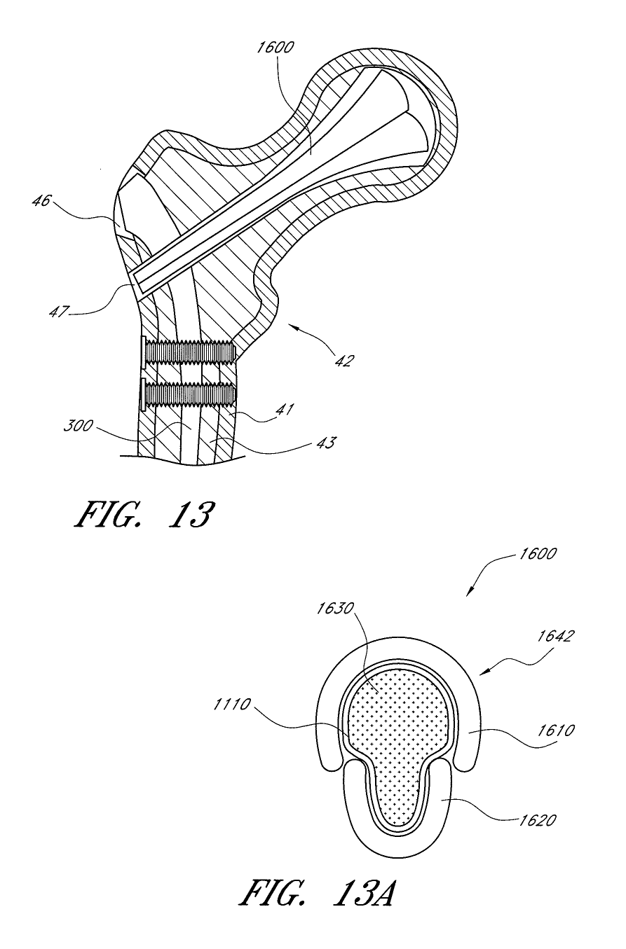

In one embodiment of a femoral fixation device 1100, an expandable tube device 1600 comprises a first elongate element 1610, a second elongate element 1620 and an expandable membrane 1630. The expandable tube device 1600 comprises a proximal segment 1602, an intermediate segment 1604, and a distal segment 1606. FIGS. 12-12B illustrate an expandable tube device 1600 in a reduced configuration 1640 according to an embodiment of the present invention. FIGS. 13-13A illustrate the expandable tube device 1600 in an expanded configuration 1642. In one embodiment the first elongate element 1610 and second elongate element 1620 have a concave interior surface and a convex exterior surface as viewed in the cross-sectional views in FIGS. 12A, 12B and 13A. In one embodiment two bores are drilled or formed to create a roughly “figure eight” shaped cavity 45 along the intermediate segment 1604 and/or distal segment 1602, as reflected in FIG. 12A. In one embodiment the expandable membrane 1630 is a balloon. In one embodiment the expandable membrane 1630 is filled with a fluid to deflect the first elongate element 1610 and second elongate element 1620 from reduced configuration 1640 to expanded configuration 1642. In one embodiment the fluid hardens. In one embodiment the fluid is a polymer. In one embodiment the fluid is fixation media 1110. In one embodiment a hardenable fluid is disposed outside the expandable tube device 1600.

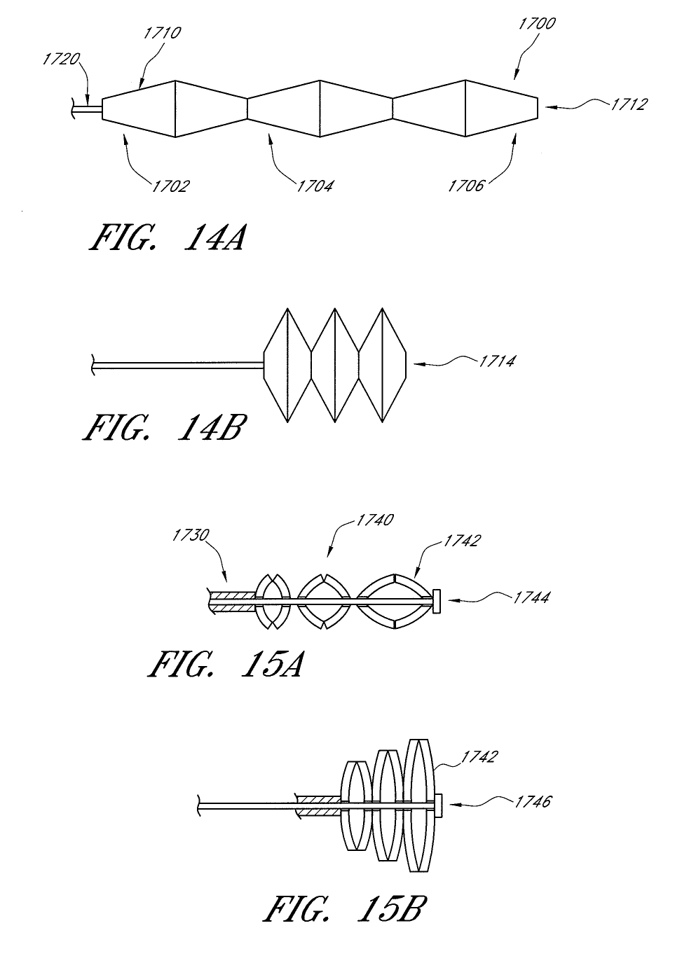

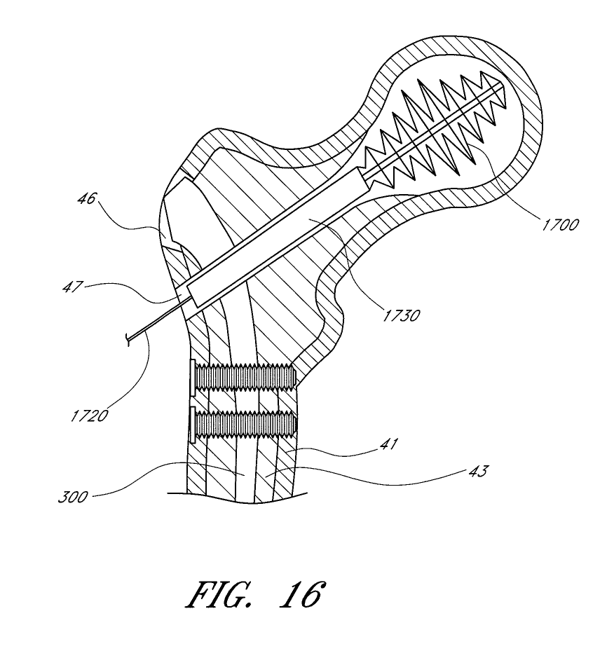

In one embodiment of a femoral fixation device 1100, a beveled bellows structure 1700 comprises one or more bellows 1710 and an elongate member 1720 with the bellows 1710 moveable between a first, radially reduced configuration 1712 and a second, radially expanded configuration 1714. In various embodiments the beveled bellows structure 1700 can include one, two, three, four, five, and up to ten, up to 15 and up to 20 or more bellows 1710. In various embodiments the bellows 1710 can be circular, oval, square, non-circular, elliptical, triangular, or other shaped in cross sectional view. The beveled bellows structure 1700 comprises a proximal segment 1702, an intermediate segment 1704, and a distal segment 1706. The proximal segment 1702, intermediate segment 1704 and distal segment 1706 do not necessarily correspond to a particular bellow 1710. FIG. 14A illustrates the beveled bellows structure 1100 in a radially reduced configuration 1712 according to an embodiment of the present invention. FIG. 14B illustrates the beveled bellows structure 1100 in a radially expanded configuration 1714. In various embodiments the elongate member 1720 can be a cable, rod, push rod, pull rod, threaded rod, tensioning element, compression element, or other structure. In one embodiment the bellows 1710 comprise a polymer.

In one embodiment the bellows 1710 is a shape memory material with spring characteristics biased to the radially reduced configuration 1712 if left free of applied forces. In one embodiment the elongate member 1720 is attached to a proximal segment 1702 of the bellows 1710. In one embodiment the elongate member 1720 can be pushed distally to apply a compressive force to the bellows 1710 to transform the beveled bellows structure 1700 to the radially expanded configuration 1714 in the femoral head 1002. In one embodiment the elongate member 1720 is attached to a distal segment 1706 of the bellows 1710 and is pulled proximally to apply a compressive force to the bellows 1710 to transform the beveled bellows structure 1700 to the radially expanded configuration 1714 in the femoral head 1002. A stop or obstruction proximal to or at the proximal segment 1702 can prevent the proximal segment 1702 from moving proximally.

In one embodiment the bellows 1710 is a shape memory material with spring characteristics biased to the radially expanded configuration 1714 if left free of applied forces. In one embodiment the elongate member 1720 is attached to a distal segment 1706 of the bellows 1710 and is pushed distally while the proximal segment 1702 of the bellows 1710 is held in place to apply tension to the bellows 1710 to transform the beveled bellows structure 1700 to the radially reduced configuration 1712 for insertion through an access hole 47 in the bone or through an anchor 1150. The bellows 1710 returns to the radially expanded configuration 1714 in the femur 1000 when the elongate member 1720 is released. In one embodiment the elongate member 1720 is attached to a proximal segment 1702 of the bellows 1710 and is pulled proximally while the distal segment 1706 of the bellows 1710 is held in place to apply tension to the bellows 1710 to transform the beveled bellows structure 1700 to the radially reduced configuration 1712 for insertion through an access hole 47 in the bone or through an anchor 1150. In one embodiment the distal segment 1706 is held distally with a push rod. In one embodiment the distal segment 1706 is held distally by anchoring the distal segment 1706 in cortical bone 41 at or near the distal end of the beveled bellows structure 1700. The bellows 1710 returns to the radially expanded configuration 1714 in the femur 1000 when the elongate member 1720 is released.

In one embodiment the bellows 1710 change shape with changes in temperature, with the radially expanded configuration 1714 corresponding to body temperature and a cooler or warmer temperature. In one embodiment the bellows 1710 is inflatable and changes shape to the radially expanded configuration 1714 as the bellows 1710 are inflated with air or some other fluid, such as fixation media 1110.

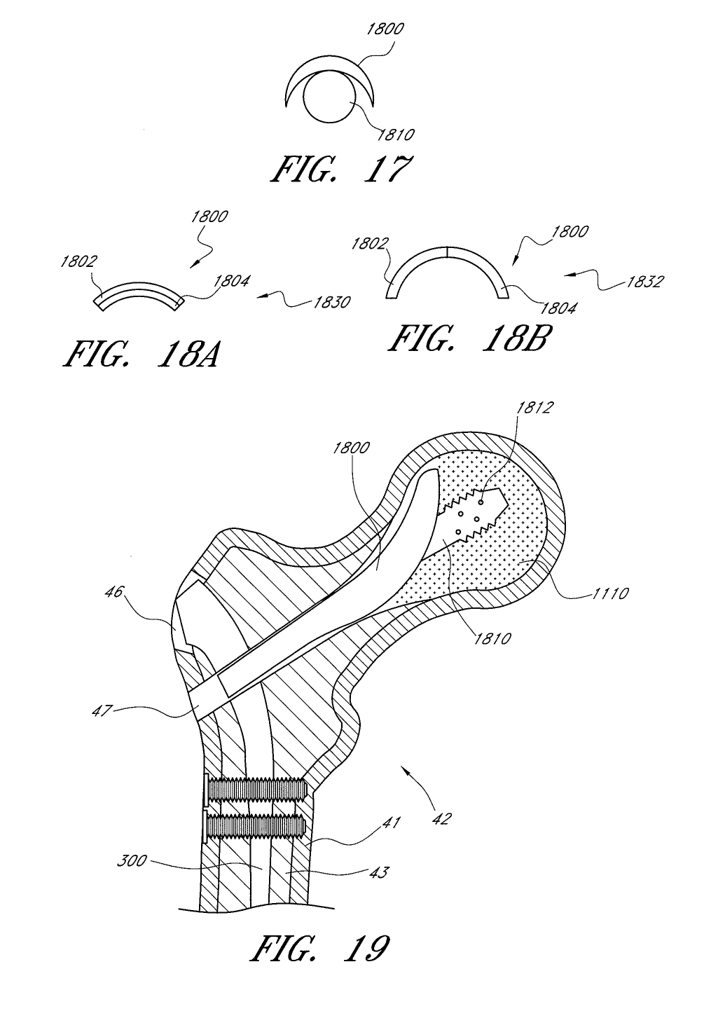

In one embodiment the beveled bellows structure 1700 further comprises one or more bevels 1740 and a slideable actuator 1730, as shown in FIGS. 15A and 15B. In one embodiment one or more bevels 1740 are disposed within a bellow 1710. In one embodiment two or more bevels 1740 have a channel for extending the elongate member 1720 through the center of the bevel 1740, with the bevels 1740 stacked such that distal motion of the slideable actuator 1730 with respect to the distal most bevel 1740 results in compression and deformation or bending of the bevels 1740 from a radially reduced configuration 1744 to a radially expanded configuration 1746. In various embodiments, varying the size, diameter, width, length, curvature, shape and interface between bevels 1740 changes the relative size, diameter, width, length, curvature and shape of the radially reduced configuration 1744 and the radially expanded configuration 1746 along the length of the elongate member 1720. In one embodiment deformation of the bevels 1740 drives the transformation of the shape of the bellows 1710.

In one embodiment of a femoral fixation device 1100, a bevel structure 1740 comprises one or more bevels 1740, an elongate member 1720 and a slideable actuator 1730. The bevel structure 1740 operates in a manner very similar to the beveled bellows structure 1700 described above, but can operate without the bellows 1710.

In one embodiment of a femoral fixation device 1100, a cortical support structure 1800 is configured to match a portion of the internal surface of the cortical bone 41 in the femoral head 1002 and/or neck 1004. FIGS. 17-19 illustrate several embodiments of a cortical support structure 1800. In one embodiment the cortical support structure 1800 is configured in a shape similar to a shoe horn. In one embodiment the cortical support structure 1800 is bio-absorbable. In one embodiment the cortical support structure 1800 comprises a unitary body. In one embodiment the cortical support structure 1800 comprises two or more members 1802, 1804 that can be moved from a reduced configuration 1830 to an expanded configuration 1832. In various embodiments the members 1802, 1804 are stackable or collapsible.

In one embodiment the cortical support structure 1800 is configured to ride along the superior or upper surface (near the cephalad side) of the femoral neck 1004 and/or femoral head 1002. In one embodiment the cortical support structure 1800 has a single radius along its cephalad surface. In one embodiment a kit of cortical support structures 1800 is provided with different shapes to conform to the variations in bony anatomy of a patient. In one embodiment the cortical support structure 1800 is similar to an elongate body 1510 described above in a shape memory cortical bone support device 1500. In one embodiment the cortical support structure 1800 has a concave cephalad or superior surface as viewed in cross section along the longitudinal axis of the cortical support structure 1800. In one embodiment the cortical support structure 1800 has a convex caudal or inferior surface as viewed in cross section along the longitudinal axis of the cortical support structure 1800.

In one embodiment the cortical support structure 1800 also comprises a support member 1810. In one embodiment the support member 1810 helps place the cortical support structure 1800 in the proper orientation with respect to cortical bone 41. In one embodiment the support member 1810 has a lumen and one or more ports 1812 for injecting fixation media 1110 in to a cavity 45 of the femur 1000. In one embodiment the support member 1810 is removed after placement of the cortical support structure 1800. In one embodiment the support member 1810 is removed after delivery of fixation media 1110. In one embodiment the support member 1810 is implanted with the cortical support structure 1800. In one embodiment the support member 1810 is a compression screw similar to embodiments of the injectable mechanical composite implant 1200. In one embodiment the support member 1810 is a lag screw to embodiments of the injectable mechanical composite implant 1200.

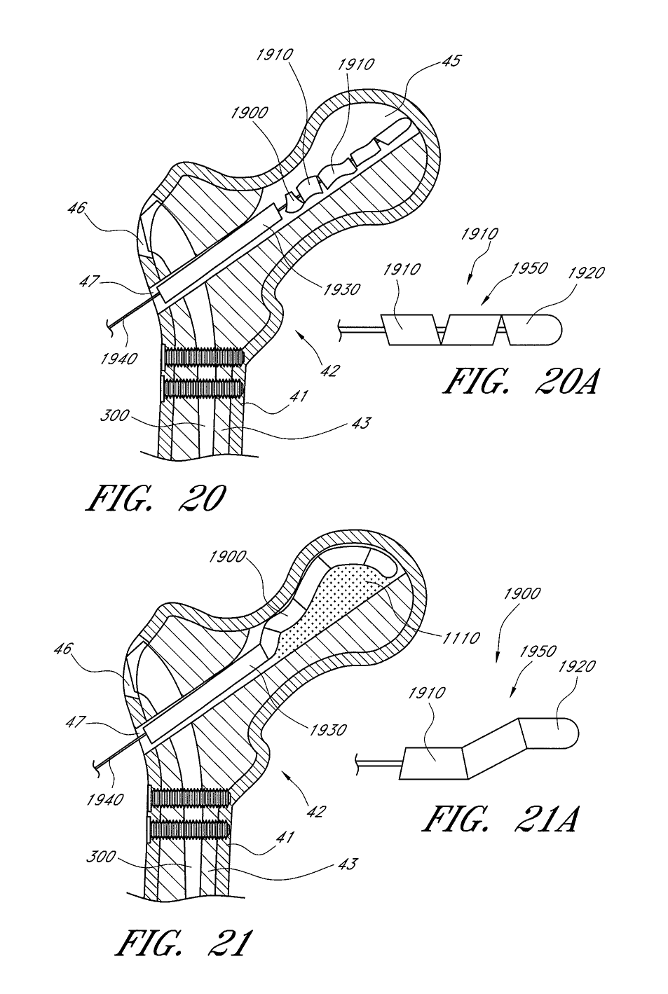

In one embodiment of a femoral fixation device 1100, a directionally actuatable cortical support structure 1900 comprises one or more support segments 1910, a distal end segment 1920, a proximal base segment 1930 and a tensile element 1940. The directionally actuatable cortical support structure 1900 is moveable between a substantially straightened configuration 1950 and an actuated configuration 1952. FIGS. 20 and 20A illustrate a directionally actuatable cortical support structure 1900 in a first, substantially straightened configuration 1950 according to an embodiment of the present invention. FIGS. 21 and 21A illustrate the directionally actuatable cortical support structure 1900 in a second, actuated configuration 1952.

In one embodiment any pair of adjacent segments 1910, 1920, 1930 can be mechanically connected to each other. In one embodiment at least one pair of adjacent segments 1910, 1920, 1930 are hingedly connected to each other. In one embodiment any of the segments 1910, 1920, 1930 comprise a string of bead-like structures. In one embodiment any of the segments 1910, 1920, 1930 comprise a tube with laser cut slots. In various embodiments the actuated configuration 1952 is configured to conform to a portion of the inner surface of the cortical bone 41 of the femoral head 1002 and/or femoral neck 1004. The tensile element 1940 is attached to the distal end segment 1920 and extends through intermediary support segments 1910, through the proximal base segment 1930 and can be placed in tension with a proximally directed force to pull the distal end segment 1920 with a force in the proximal direction. Various embodiments of complementary surfaces in the interfaces between the support segments 1910, distal end segment 1920 and/or proximal base segment 1930 are configured to move the directionally actuatable cortical support structure 1900 from the substantially straightened configuration 1950 and an actuated configuration 1952 when tension is applied to the tensile element 1940. In one embodiment the distal end segment 1920 is shaped like a spoon. In one embodiment the actuated configuration 1952 of the directionally actuatable cortical support structure 1900 is shaped like a spoon. In one embodiment fixation media 1110 is inserted around the directionally actuatable cortical support structure 1900. In one embodiment fixation media 1110 is delivered to the cavity 45 of the bone 42 through the directionally actuatable cortical support structure 1900.

In one embodiment an articulatable material delivery device 2000 comprises an articulating distal section 2010 configured to articulate within a bone 42 to deliver a material to a surface within the bone 42. FIGS. 22 and 23 illustrate an articulatable material delivery device 2000 according to an embodiment of the present invention. In one embodiment the material is fixation media 1110. In one embodiment the fixation media 1110 is a polymer. In one embodiment the articulatable material delivery device 2000 is configured for percutaneous internal resurfacing of a bone surface. In one embodiment the bone surface is an interior surface of cortical bone 41 in a femur 1110. In one embodiment cancellous bone 43 is removed and material controllably delivered by the articulatable material delivery device 2000 to reinforce a cortical bone 41 surface of interest. In one embodiment the articulatable material delivery device 2000 further comprises a heating element 2020 to warm the material being delivered. In one embodiment the heating element 2020 heats a fixation media 1110, such as a polymer, to a fluid state for percutaneous fixation media 1110 delivery. In one embodiment the polymer melts at temperatures above 50 degree Celsius. In one embodiment the polymer has a solid state that is deliverable in a rod form that can be advanced distally along a shaft 2030 in the articulatable material delivery device 2000. The polymer is heated with a heating element 2020 in to a fluid state and is delivered to a bone surface from the distal end of the articulatable material delivery device 2000. In one embodiment the articulatable material delivery device 2000 has a control mechanism 2040 for the user to manipulate for directing the articulating distal section 2010. In one embodiment the articulatable material delivery device 2000 has a trigger 2050 for advancing the fixation media 1110 distally along the articulatable material delivery device 2000. In one embodiment the articulatable material delivery device 2000 has a handle 2052 for grasping and controlling the general orientation of the articulatable material delivery device 2000. The articulatable material delivery device 2000 can be used with any of the femoral fixation devices 1100 disclosed herein. In various embodiments the fixation media 1110 can act as an anchor for a device.

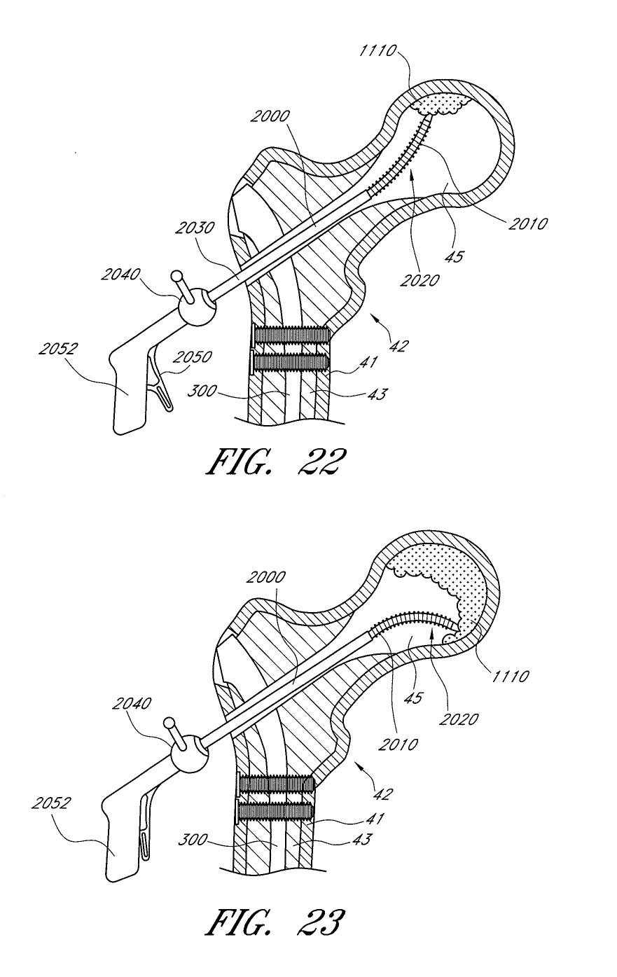

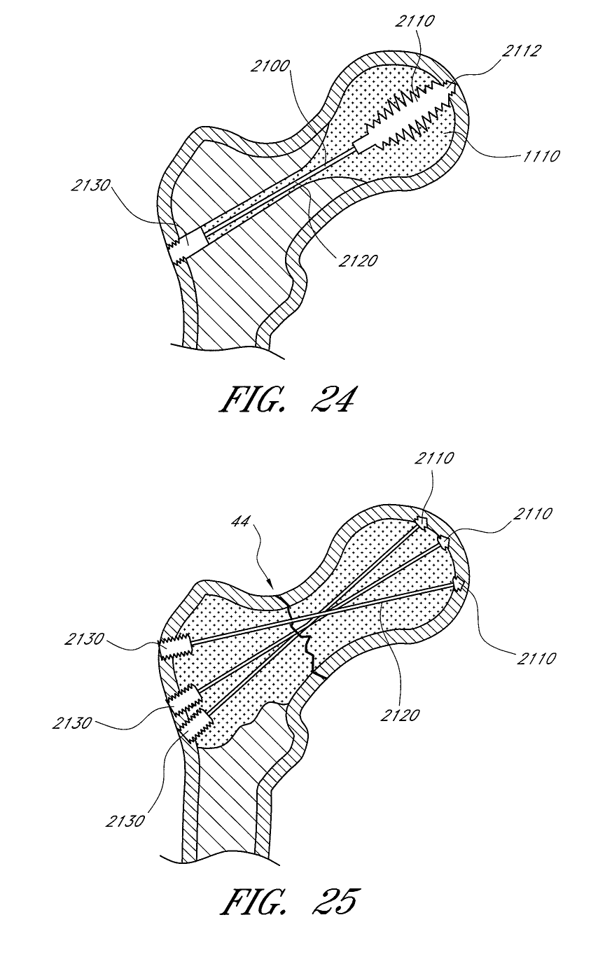

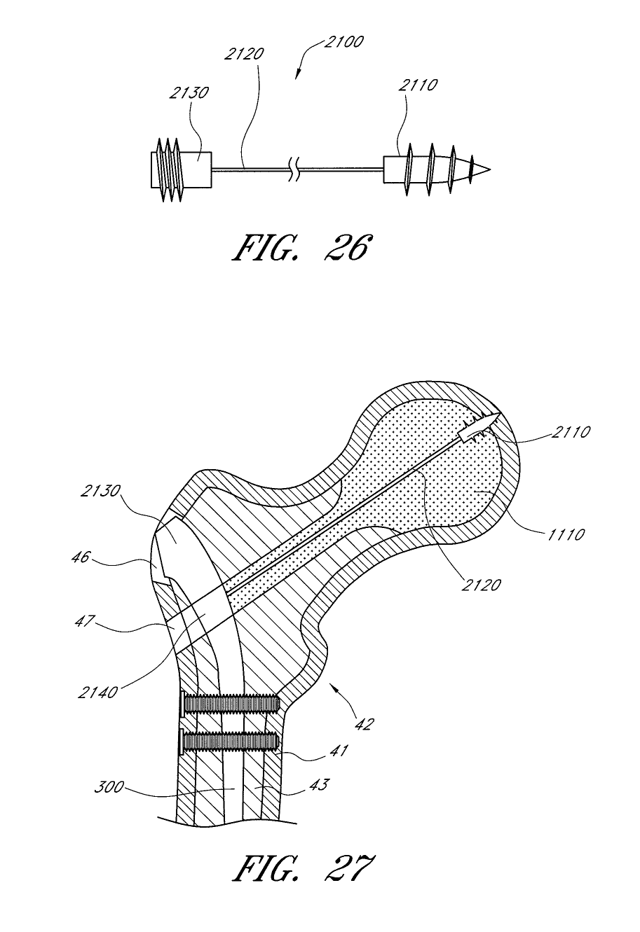

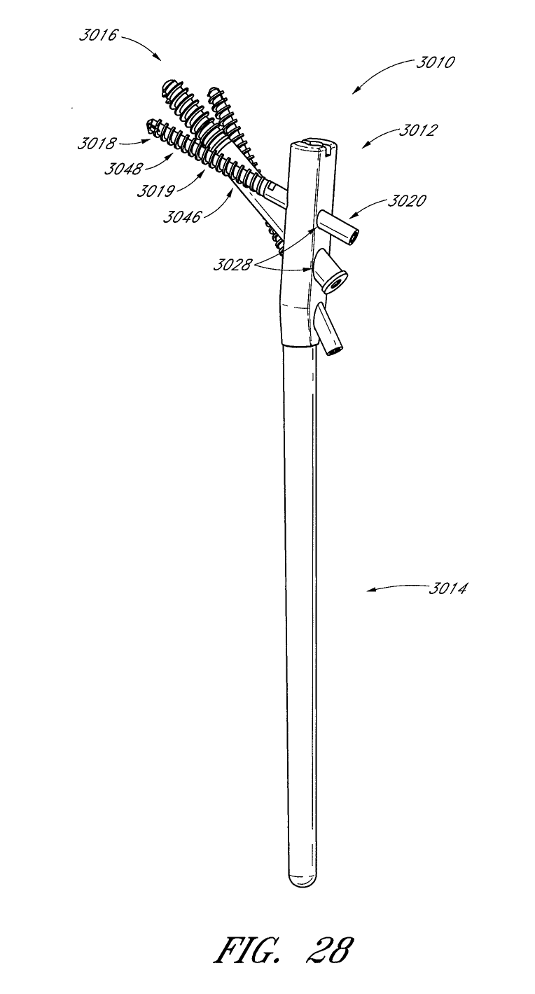

In one embodiment of a femoral fixation device 1100, an anchored cable tensioning device 2100 comprises a distal anchor 2110, a tensioning element 2120 and a proximal anchor 2130. FIGS. 24-27 illustrate various embodiment of an anchored cable tensioning device 2100. In one embodiment, the anchored cable tensioning device 2100 further comprises fixation media 1110 to fill the cavity 45 and provide support to the distal anchor 2110. In one embodiment, one or all of the components of the anchored cable tensioning device 2100 are resorbable. In one embodiment one or all of the components of the anchored cable tensioning device 2100 are resorbable to provide temporary fixation sufficient to heal a fracture 44.

In one embodiment the distal anchor 2110 is configured to maintain a position within solidified or rigid fixation media 1110 in the femoral head 1002 and/or femoral neck 1004. In one embodiment the distal anchor 2110 is held in place by fixation media 1110. In one embodiment the distal anchor 2110 does not come in contact with cortical bone 41 and is held distally by hardened fixation media 1110. In one embodiment the distal anchor 2110 comprises a cortical anchor 2112. In one embodiment the cortical anchor 2112 is configured to lock in to cortical bone 41 on the inside surface of the femoral head 1002. In one embodiment the cortical anchor 2112 is threaded. In one embodiment the cortical anchor 2112 comprises a sharp tip to pierce cortical bone 41. In one embodiment the cortical anchor 2112 is configured to lock in to cortical bone 41 without piercing or extending beyond the exterior cortical bone 41 surface on the femoral head 1002 to prevent damage to the articulation in the hip.

The tensioning element 2120 is attached to the distal anchor 2110 and proximal anchor 2130 to provide tension between the distal anchor 2110 and proximal anchor 2130, thereby imparting a compressive force to the bone 42 to assist in the healing process for injuries such as a fracture 44. In one embodiment the tensioning element 2120 is similar to an elongate member 350. In one embodiment the tensioning element 2120 is a cable. In one embodiment the tensioning element 2120 is locked at an anchor 1150. In one embodiment the tensioning element 2120 is lockable with a two-part distal collet assembly 132 as described above with respect to FIG. 38 of U.S. Provisional No. 61/150,134, filed Feb. 5, 2009, which is incorporated by reference in its entirety herein. In one embodiment the tensioning element 2120 is lockable with a cable collet anchor 272 arrangement as described above with respect to FIG. 68 of U.S. Provisional No. 61/150,134, filed Feb. 5, 2009. In one embodiment a cable tensioner assembly 200 is used to tension the tensioning element 2120.

In various embodiments the proximal anchor 2130 is a bone screw 1070, a plate 1060, or some other attachment to cortical bone 41. In one embodiment the proximal anchor 2130 is a segmented intramedullary structure 300. In one embodiment proximal anchor 2130 comprises a lock 2140. In one embodiment the proximal anchor 2130 lock 2140 allows relative motion of the tensioning element 2120 in a proximal direction only. In one embodiment the proximal anchor 2130 lock 2140 is a one way valve. In various embodiments the proximal anchor 2130 can comprise a lock to hold the tensioning element 2120. In one embodiment the proximal anchor 2130 comprises a two-part distal collet assembly 132 as described above with respect to FIG. 38 of U.S. Provisional No. 61/150,134, filed Feb. 5, 2009, which is incorporated by reference in its entirety herein. In one embodiment the proximal anchor 2130 comprises a cable collet anchor 272 arrangement as described above with respect to FIG. 68 of U.S. Provisional No. 61/150,134, filed Feb. 5, 2009. In one embodiment a cable tensioner assembly 200 is used to tension the tensioning element 2120 with the proximal anchor 2130. In various embodiments, one, two, three, four, five, up to ten, or more anchored cable tensioning devices 2100 can be deployed in a bone 42.

Turning to FIG. 28, a further embodiment of a femoral fixation device 3010 is illustrated. In one embodiment, the device 3010 comprises a head portion 3012, an intramedullary structure 3014, and plurality of elongate bodies 3016. As shown in the illustrated embodiment, the elongate bodies 3016 and the intramedullary structure 3014 can couple to the head portion 3012. In one embodiment, the intramedullary structure 3014 is configured to be placed within an intramedullary passage, such as the femoral intramedullary canal, and the elongate bodies 3016 are configured to extend through the femoral neck into the femoral head to provide support and/or compression to bone. The modular construction of the device 3010 can facilitate its placement in a patient and/or can accord flexibility in treating indications specifically.

In one embodiment, the elongate bodies 3016 have proximal ends 3020 that couple to the head portion 3012 through a plurality of apertures 3028. In one embodiment, the apertures are through holes within the head portion 3012. In one embodiment, the apertures 3028 are angled such that the elongate bodies 3016, when coupled with the apertures 3028, are angled with respect the head portion 3012 and/or each other in at least one plane. In one embodiment, the elongate bodies 3016 are angled such that their distal ends 3018 diverge from each other in a divergence zone 3048. In one embodiment, the elongate bodies 3018 are angled such that the middle portions 3019 of the elongate bodies 3016 converge in a convergence zone 3046. In one embodiment, the convergence zone 3046 facilitates passing the elongate bodies 3016 through the relatively narrow femoral neck and the divergence zone 3048 is configured to be located within the relatively larger femoral head to facilitate, among other functions, bridging and/or compressing a femoral fracture.

In operation, one embodiment of the intramedullary structure 3014 and head portion 3012 are preferably installed in an intramedullary passage, for example a femur intramedullary canal, with the head portion 3012 disposed near the top, or cephalad, thereof. In one embodiment, incisions in the skin and tissue are made corresponding to the locations of the plurality of apertures 3028 on the head portion 3012. In one embodiment one or more holes corresponding to one or more of the apertures 3028 are bored into the osseous structure. In one embodiment the elongate bodies 3016 are inserted through the incisions and into the plurality of apertures 3028. In one embodiment the proximal ends 3020 are coupled to the head portion 3012 by way of the apertures 3028. In one embodiment, the distal ends 3018 extend into the holes bored into the osseous structure. In one embodiment the elongate bodies 3016 extend through a narrow osseous structure, such as the femoral neck, and the distal ends 3018 expand into a larger osseous structure, such as the femoral head. In one embodiment the elongate bodies 3016 are coupled to both the head portion 3012 and osseous tissue. In one embodiment the elongate bodies impart a compressive force to the osseous tissue to compress and/or support a fracture. In one embodiment, the divergence of the distal ends 3018 within an osseous structure inhibits unintentional removal of the elongate bodies 3016.

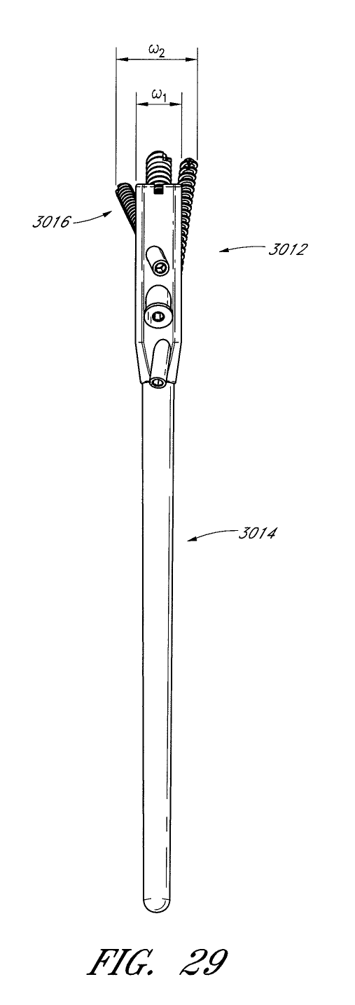

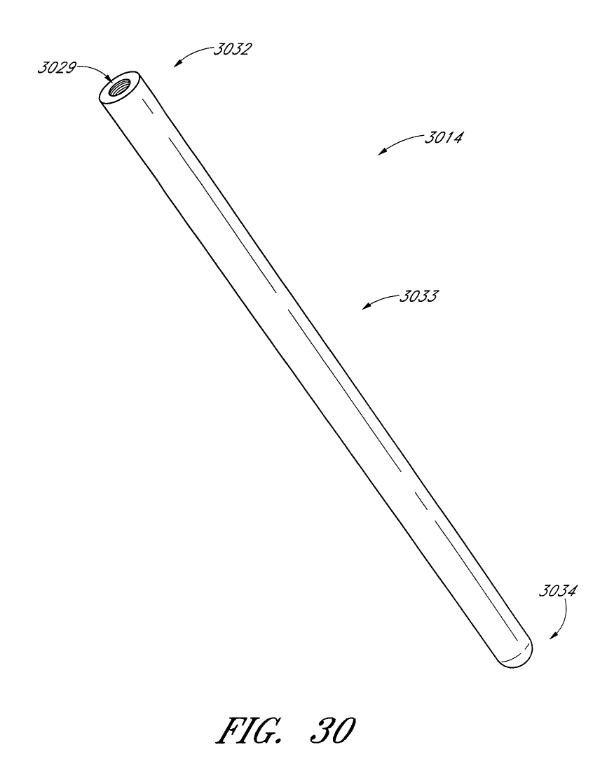

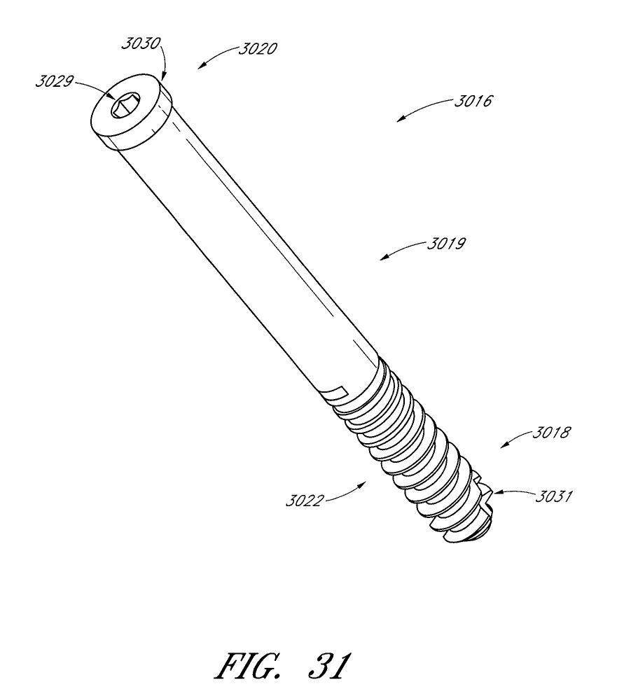

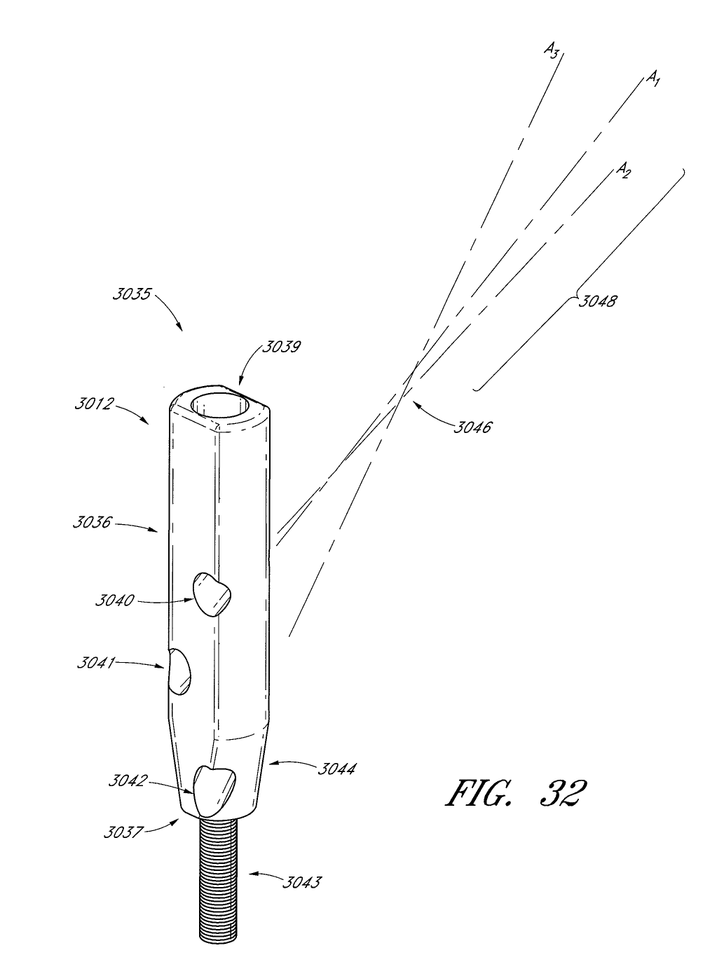

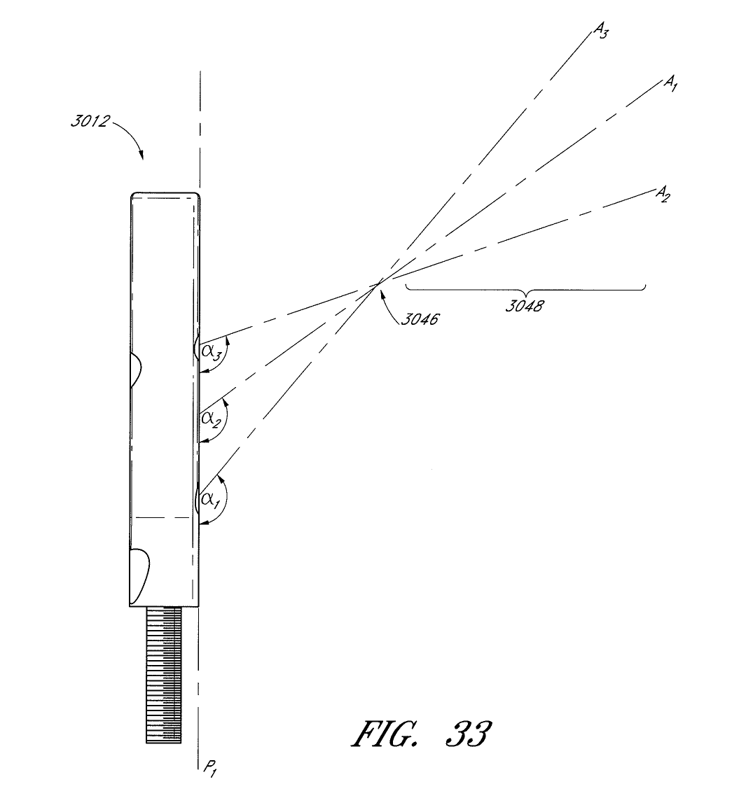

FIG. 29 illustrates an embodiment of the divergence of the elongate bodies 3016 as compared to the head portion 3012. In this embodiment, the proximal ends 3020 of the elongate bodies 3016 are about co-planar in a plane parallel to the longitudinal centerline of the head portion 3012. In comparison, the distal ends 3018 of this embodiment diverge into disparate longitudinal planes. In one embodiment, the distal ends 3018, diverge into disparate horizontal planes. In some embodiments the distal ends 3018 can define a width W2 that is greater than the width W1 of the head portion 3012.

With reference to FIG. 30, an embodiment of the intramedullary structure 3014 is illustrated. In one embodiment, the intramedullary structure 3014 comprises a base 3032, a middle portion 3033, and a distal end 3034. In one embodiment, the intramedullary structure 3014 is constructed from a biocompatible material, for example but not limited to, titanium, stainless steel, or tungsten. In one embodiment the intramedullary structure 3014 is rigid, to facilitate, for example, support to osseous tissue. In one embodiment the intramedullary structure 3014 is flexible, to facilitate, for example, placement of the structure 3014 within an intramedullary passage. In one embodiment the intramedullary structure 3014 is a polymer. In one embodiment intramedullary structure 3014 is constructed from PEEK. In one embodiment the intramedullary structure 3014 is segmented, as described herein or in any of the descriptions of segmented intramedullary structures incorporated by reference, herein, from U.S. Provisional No. 61/180,342, filed May 21, 2009, U.S. application Ser. Nos. 12/345,451, 12/345,225 and 12/345,340 (all of which were filed Dec. 29, 2008 as continuations-in-part of U.S. application Ser. No. 12/052,919, filed Mar. 21, 2008), and U.S. application Ser. No. 12/052,919, filed Mar. 21, 2008, which claim the benefit of priority from U.S. Provisional No. 60/896,342 filed Mar. 22, 2007.

In one embodiment, the base 3032 is configured to couple to the head portion 3012. The base 3032 can comprise an aperture 3029 configured to receive a corresponding portion of the head portion 3012. For example, in one embodiment, the base aperture 3035 includes a smooth hole, which is sized and shaped to receive a corresponding portion of the head portion 3012. In another embodiment the base aperture 3029 is configured with female threads to receive a corresponding male-threaded portion of the head portion 3012. In another embodiment the base aperture 3029 is configured with male threads to receive a corresponding female-threaded portion of the head portion 3012. In another embodiment the base aperture 3029 has a hexagonal aperture to receive a suitably-sized hexagonal portion of the head portion 3012. In yet a further embodiment, the base 3032 comprises a stem that is receivable and coupleable to a corresponding aperture in the head portion 3012. In various embodiments, a variety of techniques for coupling the intramedullary structure 3014 and head portion 3012 are possible, such as but not limited to threads, interference fit, chemical bonding agents such as glue, combinations thereof, or the like. In yet further embodiments, the intramedullary structure 3014 and the head portion 3012 are a single piece.

In some embodiments, the intramedullary structure is 3014 angled with respect to the head portion 3012. For instance, in one embodiment the intramedullary structure 3014 is angled about 1-10° with respect to the longitudinal centerline of the head portion 3012. In another embodiment the intramedullary structure 3014 is angled is about 5-7° with respect to the longitudinal centerline of the head portion 3012. Among other advantages, such angling can facilitate placement of the intramedullary structure 3014 within the intramedullary passage.

The shape of the intramedullary structure 3014, in the illustrated embodiment, is a cylinder that tapers from the base 3032 to the distal end 3034. However, other embodiments are non-tapered and/or non-cylindrical. Embodiments of the intramedullary structure 3014 can include a feature, such as a ridge or a groove, disposed along some or all of the longitudinal length of the structure. Some embodiments have a shoulder and/or a ring. For example, one embodiment of the intramedullary structure 3014 has a shoulder located about 125 mm from the base, as measured along the longitudinal length of the structure. In one embodiment, such a shoulder reduces the diameter of the intramedullary structure 3014 about 1 mm from the shoulder to the distal end 3034.

The middle portion 3033 of the intramedullary structure 3014 can comprise a variety of shapes. In one embodiment the middle portion 3033 includes a substantially straight portion. In one embodiment the middle portion 3033 comprises a curved portion. In yet another embodiment the middle portion 3033 comprises substantially straight and curved portions. In one embodiment, the middle portion 3033 conforms to the shape of the intramedullary passage in which the intramedullary structure 3014 is placed.

In the illustrated embodiment in FIG. 30, the distal end 3034 is rounded. In other embodiments, the distal end 3034 is conical, frustocontical, or blunt. In still further embodiments the distal end 3034 can be expanded, as will be discussed in further detail below. In a preferred embodiment, the distal end 3034 can be fixed to osseous tissue. In one embodiment, the distal end 3034 can comprise a radiographic marker. In one embodiment, the marker is annular.

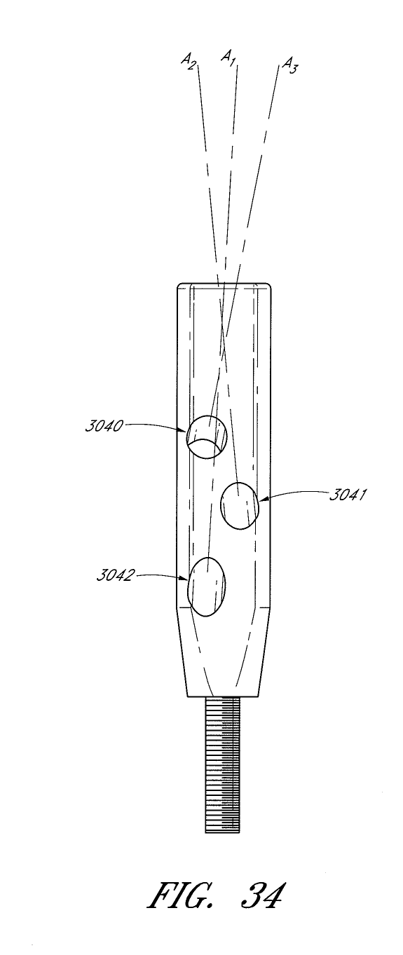

With reference to FIG. 31, an embodiment of an elongate body 3016 is depicted. In one embodiment, the elongate body comprises a distal end 3018, a middle portion 3019, and a proximal end 3020. In one embodiment the elongate body 3016 has a threaded section 3022. In the illustrated embodiment thread are located at the distal end 3018. However, other embodiments may have threads located at the middle portion 3019 or proximal end 3020. Yet further embodiments have a combination of thread locations, for example an embodiment of the elongate body 3016 has threads at the distal end 3018 and proximal end 3020, but is smooth in the middle portion 3019. Still other embodiments are unthreaded.

Various embodiments employ a variety of types of elongate bodies 3016. For example, in some embodiments the elongate bodies 3016 are rods, spikes, screws, molly-bolts, cams, nails, combinations thereof, or the like. In one preferred embodiment, the elongate bodies 3016 are lag bolts. In some embodiments, the elongate bodies 3016 are of the same type, such as all being lag bolts. However, in other embodiments, each of the elongate bodies 3016 is a different type. For instance, one embodiment has a nail, a rod, and has a screw for elongate bodies 3016. Some embodiments employ a combination of types of elongate bodies 3016. For example, in one embodiment, one elongate body is a lag bolt and two elongate bodies are screws. In one embodiment one elongate body is a molly-bolt and another elongate body is a textured rod.

A variety of embodiments employ elongate bodies 3016 with an assortment of sizes and shapes. In various embodiments, the elongate bodies 3016 have cross-sectional shapes such as but not limited to, circular, rectangular, square, octagonal, or similar. In one embodiment, the elongate bodies 3016 are straight, although other embodiments utilize curved elongate bodies 3016. In some embodiments the elongate bodies 3016 are the same diameter; however other embodiments have elongate bodies with disparate diameters. In still further embodiments, some of the elongate bodies have the same diameter, while others are a different diameter. In one embodiment, the elongate bodies have a diameter of 3-15 mm. In one embodiment, the elongate bodies have a diameter of 6-10 mm. For example, in one embodiment two elongate bodies have a 6.35 mm diameter and one elongate body has a 9.5 mm diameter. A variety of lengths for the elongate bodies is also contemplated. In one embodiment, the elongate bodies have lengths of 60-120 mm. In one embodiment, the elongate bodies have lengths of 80-100 mm. For example, in one embodiment, one elongate body is about 100 mm long, while at least one other elongate body is about 85 mm long.

The elongate bodies 3016 are preferably configured to couple to apertures 3016 in the head portion 3012. In one embodiment this coupling is achieved by a threaded connection. In one embodiment the coupling occurs by way of a press fit. In one embodiment a bonding agent or operation, such as glue or welding, achieves the coupling. In one embodiment, one or more of the elongate bodies 3016 are integrally formed with the head portion 3012.

The elongate bodies 3016 preferably include features to facilitate insertion. For example, in the embodiment shown, the elongate body 3016 is provided with an aperture sized and configured to receive a hexagonal wrench, by which a torque may be applied to the elongate body 3016. It will be understood that various other insertion features are contemplated, such as a flat-head screw, Philips-head screw, square-head screw, or similar. The illustrated embodiment further includes a flange 3030 insertion feature. The diameter of the flange 3030 preferably is greater than the diameter of the aperture 3028 through which the elongate body 3016 couples, thereby, for example, serving as a physical stop and/or inhibiting over-insertion of the elongate member 3016. Some embodiments have insertion features at the distal end 3018. For example, in the embodiment shown, the threaded portion 3022 at the distal end 3018 has one or more cutting threads 3031 to facilitate threading the elongate body 3016 into osseous material.

Turning to FIGS. 32-35, an embodiment of a head portion 3012 is illustrated. In the illustrated embodiment, the head portion 3012 comprises an upper portion 3035, a body 3036, a lower portion 3037, and a plurality of apertures 3040-42. In one embodiment, the head portion 3012 is sized and configured to be placed within an intramedullary passage, such as the femoral intramedullary canal. The head portion 3012 is preferably sufficiently rigid to support the loads and stresses imposed by the elongate bodies 3016 and the intramedullary structure 3014. The head portion 3012 is preferably constructed from a biocompatible material, for example but not limited to, titanium, stainless steel, or tungsten. In one embodiment, the head portion 3012 is constructed from a polymer. In one embodiment, the head portion 3012 is constructed from PEEK.

The upper portion 3035, in one preferred embodiment, comprises dorsal aperture 3039. In one embodiment, the dorsal aperture 3039 is parallel to the longitudinal axis of the head portion 3012. In one embodiment the aperture is positioned at the longitudinal centerline of the head portion 3012, but in other embodiments the dorsal aperture 3039 is offset from this centerline by, for example, 0-2.5 mm. In one embodiment, the dorsal aperture 3039 extends through a portion of the head portion 3012 and interconnects with one or more of the plurality of apertures 3040-42 that couple to the elongate bodies 3016. In some embodiments the diameter of the dorsal aperture 3039 is greater than the diameter of the apertures for the elongate bodies 3016. In one embodiment the dorsal aperture 3039 can be used to secure and/or facilitate insertion of one or more of the elongate bodies 3016. For example, in one embodiment, an agent, such as glue, lubricant, and/or coolant, is injected into the dorsal aperture 3039. In one embodiment, the dorsal aperture 3039 includes threads to receive a set-screw. Further, some embodiments have a feature in the upper portion 3035 to facilitate placement of the head portion 3012 into the intramedullary space. For instance, one embodiment comprises a vertical slot (not shown) in the upper portion 3035, which is configured to receive a tool to aid in installing the head portion 3012.

In the illustrated embodiment, the body 3036 is shaped with two opposed substantially flat vertical faces joined by two rounded vertical faces. Other embodiments have alternate body 3036 shapes, such as cylindrical, square, rectangular, or otherwise. Any size and shape configured to fit in an intramedullary passage is possible.

In the illustrated embodiment, the body 3036 comprises the apertures 3040-42 and from each aperture 3040-42 extends a respective axis A₁-A₃. Thus, aperture 3040 corresponds to axis A₁, aperture 3041 corresponds to axis A₂, and aperture 3042 corresponds to axis A₃. When straight elongate bodies 3016 are coupled with the head portion 3012 through the plurality of apertures 3040-42, the elongate bodies preferably extend along the axes A₁-A₃. In the illustrated embodiment, the axes A₁-A₃ converge in a convergence zone 3046 and diverge in a divergence zone 3048. In one embodiment, when coupled with the apertures 3040-42, the elongate bodies 3016 converge in the convergence zone 6034 and diverge in the divergence zone 3048. In some embodiments, the elongate bodies 3016 remain spaced apart. In other embodiments, at least two of the elongate bodies 3016 contact. In some embodiments, at least two of the elongate bodies 3016 contact in the convergence zone 3046.

Various embodiments employ a variety of aperture 3040-42 configurations. The illustrated embodiment comprises three apertures 3040-42. Other embodiments comprise two, four, five, or more apertures 3040-42. In one embodiment, the cross-section of the apertures 3040-42 is similarly shaped to the cross-section of the corresponding elongate bodies 3016. In some embodiment, the diameter of the apertures 3040-42 is slightly larger, such as 0.25 mm, than the elongate bodies 3016 to be received. In some embodiments the diameter of the apertures 3040-42 is slightly smaller, such as 0.25 mm, than the elongate bodies 3016 to be received. In one embodiment the apertures 3040-42 have a diameter of 3-15 mm. In one embodiment the apertures 3040-42 have a diameter of about 6-10 mm. In one embodiment, the apertures 3040-42 are approximately in line with the longitudinal centerline of the head portion 3012. In one embodiment, the apertures 3040-42 are on one side of the longitudinal centerline of the head portion 3012. In one embodiment, the apertures 3040-42 are on both sides of the longitudinal centerline of the head portion 3012. In one embodiment, the apertures 3040-42 are spaced about 3-15 mm apart. In one embodiment, the apertures 3040-42 are spaced about 5-10 mm apart.

As discussed above, the elongate bodies 3016 may be configured to couple to the apertures 3040-42. Similarly, the apertures 3040-42 preferably are configured to receive and couple to the elongate bodies 3016. For instance, in one embodiment, the elongate bodies 3016 have male threads and the apertures 3040-42 have corresponding female threads. In one embodiment the apertures 3040-42 are of such diameter as to slideably receive the elongate bodies 3016. Various ways of coupling the elongate bodies 3016 and the head portion 3012 are possible, such as threads, press fit, adhesive, glue, or the like. In some embodiments, one or more apertures 3040-42 do not receive an elongate body 3016.

The lower portion 3037 of the head portion 3012 preferably couples to the base 3032 of the intramedullary structure 3014. In the illustrated embodiment, the lower portion 3037 has a stem 3043 which can be configured to be receivable within the aperture 3029 of the base 3032 of the intramedullary structure 3014. As shown, the stem 3043 can be a smooth cylinder, but in other embodiments the stem 3043 is threaded and/or has a cross-section that is rectangular, hexagonal, elliptical, star-shaped, or the like. In one embodiment the stem 3043 has a blunt end, while in other embodiments the stem 3043 has a conical or rounded end. The illustrated embodiment has a taper 3044, however other embodiments are untapered.

As shown in the embodiment illustrated in FIG. 33, the axes A₁-A₃ can each have an angle of elevation α₁-α₃ with respect to the plane P₁. In one embodiment, the angles of elevation α₁-α₃ are 20°-140° with respect to a plane P₁ along the rear face of the head portion 3012. In one preferred embodiment, angle αₗ corresponding to axis Aₗ has an angle of elevation of about 110°, α₂ corresponding to axis A₂ has an angle of elevation of about 128° and α₃ corresponding to axis A₃ has an angle of elevation of about 140°. In another embodiment, αₗ has an angle of about 125°, α₂ has an angle of about 85° and α₃ has an angle of about 135°. In a further embodiment, the angles α₁-α₃ are all about 130°. Various other angles of the angles of elevation α₁-α₃ are possible.

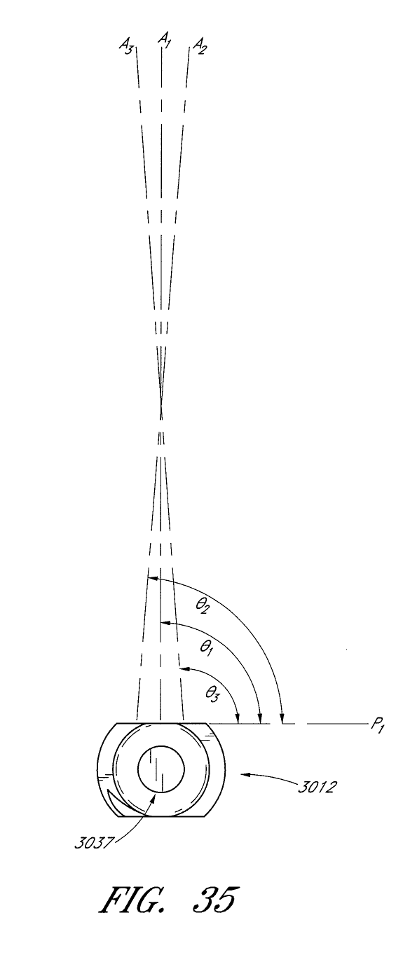

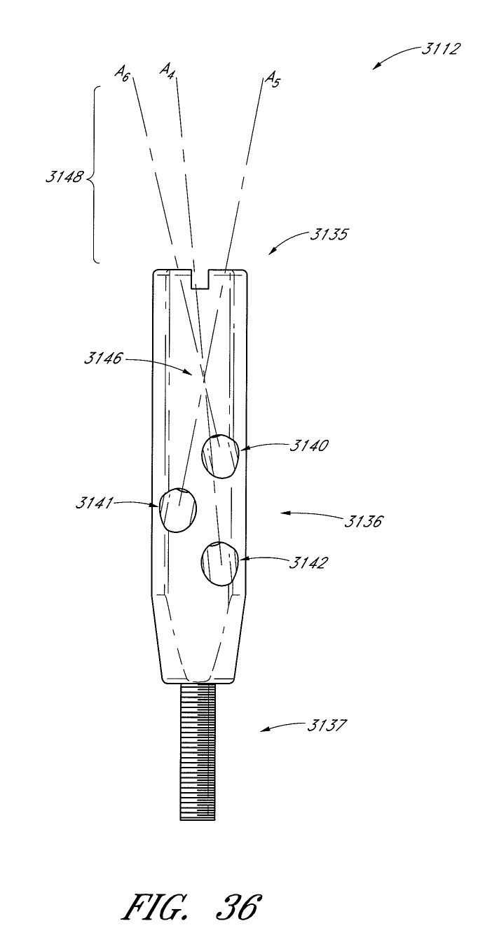

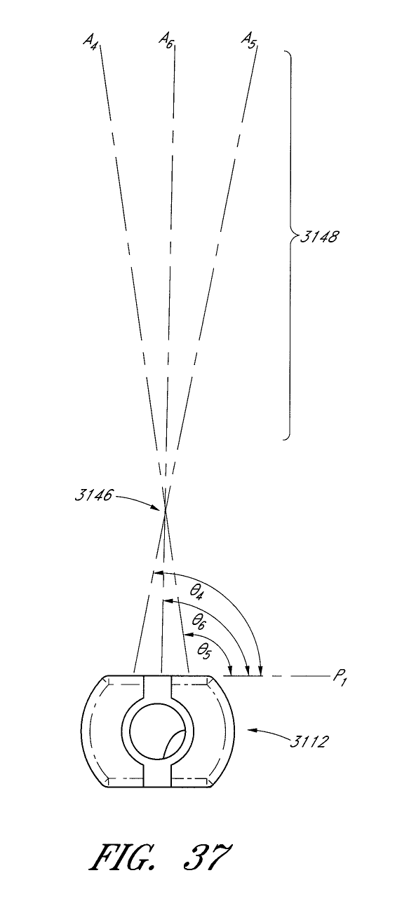

As shown in the embodiment illustrated in FIG. 35, the axes A₁-A₃ can each have an angle of deflection θ₁-θ₃ with respect to the plane P₁. The angles of deflection θ₁-θ₃ are preferably between 20°-160° with respect to the plane P₁. For example, in the embodiment shown, angle θ₁ corresponding to the axis Aₗ is about 90°, while angles θ₂ and θ₃, corresponding to axes A₂ and A₃ respectively, are about 80° and 100°, respectively. Various other angles of elevation are contemplated.

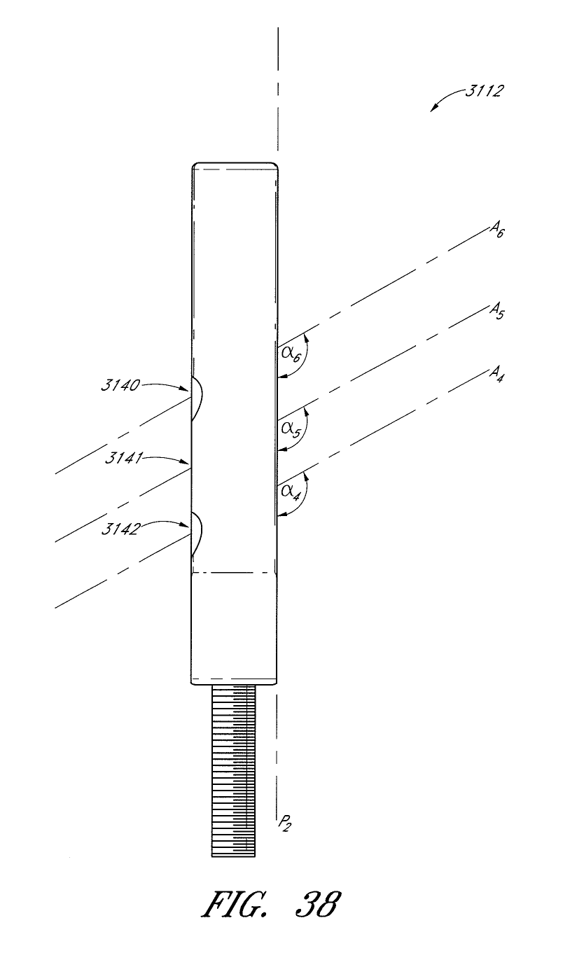

Turning to FIGS. 36-38, another embodiment of a head portion 3112 is illustrated. In one embodiment, this embodiment can be coupled to the intramedullary structure 3014 and elongate bodies 3016 as described above. The head portion 3112 can comprises an upper portion 3135, a body 3136, a lower portion 3137, and two or more apertures 3140-42. In one embodiment, the elongate bodies 3016 are coupled to the head portion 3112 through the apertures 3140-42 and extend along axes A₄-A₆ that correspond to the centerline of the apertures 3140-22. As depicted in FIGS. 36 and 37, in some planes the axes A₄-A₆ can converge in a convergence zone 3146 and diverge in a divergence zone 3148. As discussed above, when used for femoral fixation, preferably the convergence zone 3146 is located in the femoral neck and the divergence zone 3148 is located in the femoral head.

As shown in FIG. 38, in some embodiments the axes A₄-A₆ can be parallel in at least one plane. In such embodiments, the angles of elevation, α₄-α₆ with respect to a plane P₂ are about equal. For example, in one embodiment the angles of elevation α₄-α₆ are each about 120°. In another embodiment, α₄-α₆ are each about 100°. Various parallel angles of elevation α₄-α₆ are contemplated, but 20°-140° is preferred. Further, in some embodiments the angles of deflection θ₄-θ₆ with respect to the plane P₂ are about equal. In one embodiment, each angle of deflection θ₄-θ₆ is about 90°. Various embodiments utilize a variety of parallel angles of deflection θ₄-θ₆, but 20°-160° preferred.

With reference to FIGS. 39-41, another embodiment of a head portion 3212 is illustrated. This embodiment preferably couples to the intramedullary structure 3014 and elongate bodies 3016 discussed above. As shown, the head portion 3212 can comprises an upper portion 3235, a body 3236, a lower portion 3237, and two or more apertures 3240-42. The elongate bodies 3016 preferably couple to the head portion 3212 through the apertures 3240-42 and extend along the apertures' corresponding axes A₇-A₉.

Moreover, in this “hybrid” embodiment, the apertures 3240-42 themselves can have more than one aperture. For example, the upper aperture 3240 can comprise a right and a left aperture 3240R, 3240L. In one embodiment, the centerline of the apertures 3240R, 3240L are non-parallel. As shown, in some embodiments the apertures 3240R, 3240L interconnect. Similarly, in one embodiment, the lower aperture 3242 comprises a right and a left aperture 3242R, 3242L. Such multiple apertures can provide, among other benefits, the advantage of being able select among multiple positions and/or angles when coupling an elongate body 3016 to the head portion 3212. Such flexibility can also provide the advantage of being able to use the same head portion 3212 embodiment on both the right and left side of the body.

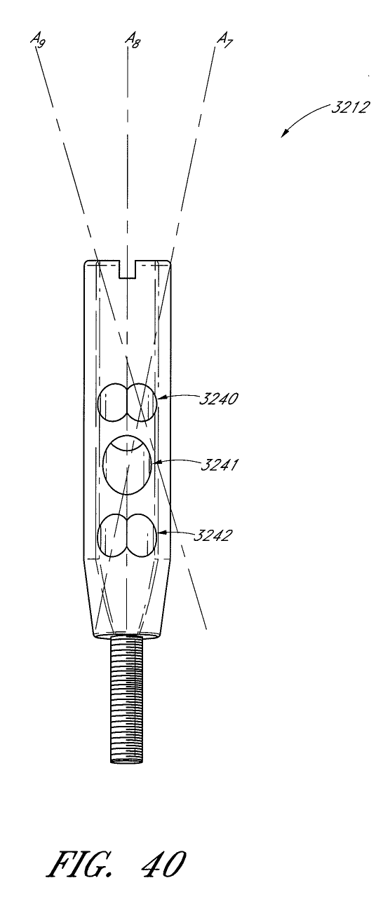

Various embodiments employ a variety of combinations of apertures 3240-42. In the embodiment shown in FIG. 40, the upper aperture 3240 and lower aperture 3242, each have right and left apertures, while the middle aperture 3241 has only one aperture. In one embodiment, the middle aperture 3241 has right and left apertures, while the upper and lower apertures 3240, 3242 each have a single aperture. In one embodiment, each of the apertures 3240-42 each have a left and right aperture. In one embodiment, at least one of the apertures 3240-42 has a top aperture and a bottom aperture. In one embodiment, at least one of the apertures 3240-42 has a top-right aperture and a bottom-left aperture. In one embodiment, at least one of the apertures 3240-42 has a top-left aperture and a bottom-right aperture. Other embodiments have other combinations of hybrid apertures.

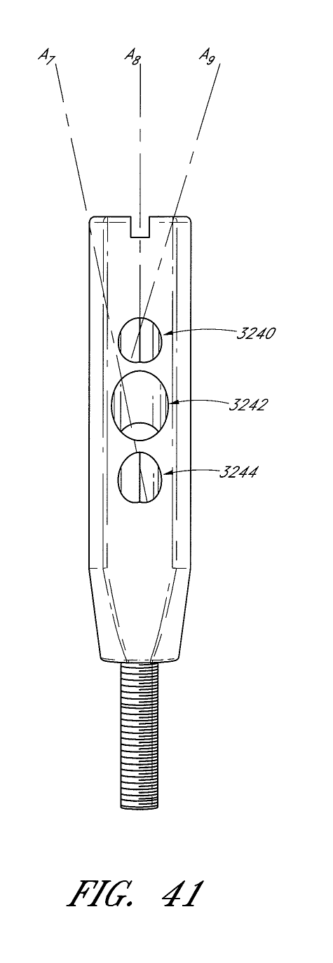

In some embodiments some apertures interconnect and/or merge as they pass through the head portion 3212. For example, the rear view of the illustrated embodiment, as shown in FIG. 41, has only a single exit for aperture 3240-42. Thus in the illustrated embodiment, certain apertures (3240R and 3240L, 3242R and 3242L) merged in the course of passing from the front to the rear of the head portion 3212. In another embodiment the apertures remain distinct, even though they can interconnect with other apertures.

Further, some embodiments provide the ability to choose the divergence pattern of the elongate bodies 3016. For instance, in one embodiment, coupling the elongate bodies 3016 in a certain selection of apertures 3240-42 results in the elongate bodies being parallel in at least one plane, but coupling the elongate bodies 3240-42 in different selection of apertures 3240-42 results in the elongate bodies 3016 being non-parallel in all planes.

Turning now to FIG. 42, another embodiment of an intramedullary structure is illustrated. In various embodiments, the intramedullary structure is adapted to be received in the intramedullary canal of a bone, such as a long bone, including but not limited to a femur, tibia, fibula, humerus, radius, ulna, phalange, metatarsal, metacarpal, clavicle or other long bone. In one embodiment the intramedullary structure comprises a substantially straight portion. In one embodiment the intramedullary structure comprises a curved portion. In one embodiment the intramedullary structure comprises substantially straight and curved portions. In one embodiment, the intramedullary structure comprises a plurality of segments. In various embodiments, different combinations of segments can be used or combined in a modular fashion to assemble custom made structures based on the bone and application for the structure. In some embodiments the intramedullary structure is removable from the body. In various embodiments the overall configuration or shape of the intramedullary structure may be straight, substantially straight, or curved along any one segment or any sets of segments. Each segment can be substantially straight or curved, and any set of straight segments can have interfaces providing for angles between adjacent segments. In one embodiment the intramedullary structure has a first configuration and a second configuration. In one embodiment the first configuration is substantially the configuration of the intramedullary structure once it is assembled and delivered into the intramedullary canal. In one embodiment the second configuration is the configuration of the intramedullary structure once it is locked. In one embodiment an intramedullary structure configuration is linear. In one embodiment an intramedullary structure configuration is substantially linear. In one embodiment an intramedullary structure configuration is curved. In one embodiment an intramedullary structure configuration is predetermined. In one embodiment a predetermined configuration mimics the contour of the intramedullary canal. In one embodiment an intramedullary structure configuration is governed by the native structure of the intramedullary canal in which the structure is inserted. In one embodiment an intramedullary structure configuration conforms to the structure of the surrounding tissue. In one embodiment an intramedullary structure configuration is flexible. In one embodiment an intramedullary structure configuration is substantially rigid. In one embodiment an intramedullary structure configuration is rigid. In one embodiment an intramedullary structure can change from a relatively longer configuration to a relatively shorter configuration. In one embodiment an intramedullary structure configuration is movable within one plane. In one embodiment an intramedullary structure configuration is movable in two planes. In one embodiment an intramedullary structure configuration is movable in three or more planes. In one embodiment an intramedullary structure configuration is axially compressible. In one embodiment an intramedullary structure configuration is rotatable about a longitudinal axis. In one embodiment an intramedullary structure configuration is axially rotatable. In one embodiment an intramedullary structure configuration is locked.

Various embodiments of an intramedullary structure as disclosed herein may list various parameters, such as sizes, lengths, diameters, widths, curvatures and geometry that can conform to or be implanted based on various parameters of bones and of structures in which embodiments of the devices may be configured to be implanted. Listings provide some examples, but should not be read to limit the disclosure to those specific dimensions or characteristics. For example, dimensions of an intramedullary structure and its various size and shape and feature characteristics can vary depending on parameters of the bone and/or patient, the type of fracture, and other factors. Embodiments of intramedullary structures are scalable. For example, some non-limiting diameters (or widths) of certain embodiments of an intramedullary structure could range from about 5 mm (for such uses as pediatric bones, or adult clavicle, radius) to about 18 mm (for such uses as an adult femur). Embodiments of lengths of an intramedullary structure could very from a few inches to 800 mm in a knee fusion nail (from ankle to hip). Various embodiments may be configured for implantation in any long bone anatomies, including but not limited to a femur, tibia, fibula, humerus, ulna, radius, clavicle, metatarsals, metacarpals, and others.

In various embodiments, any of the disclosed embodiments of parts and ranges of sizes, angles, dimensions, or otherwise may be provided in a kit. In an embodiment, components can be combined or assembled from a modular kit.

In one embodiment, an intramedullary structure is configured for insertion in a femoral bone. In various embodiments the femoral intramedullary structure can be provided in various diameters, such as (but not limited to) about 8-18 mm. In various embodiments the femoral intramedullary structure can be provided in various lengths, such as (but not limited to) about 170-500 mm. In other embodiments, the intramedullary structure can be of sufficient length to span from about the ankle to about the hip, such as (but not limited to) about 250-1000 mm. In various embodiments the femoral bone screws can have a diameter of about 4-12 mm and lengths such as (but not limited to) about 60-150 mm. In one embodiment the bone screws can have a 6.35 mm diameter and a 100 mm length. In one embodiment the bone screws can have a 9.5 mm diameter and a 110 mm length.

In various embodiments an intramedullary structure is configured for insertion in bones of varying shapes and/or sizes. In various embodiments, the nominal diameter of an intramedullary structure can be about 8-18. In various embodiments, the diameter of a proximal end of an intramedullary structure can be about 8-18 mm or another dimension. In various embodiments, transition portions can range in width or diameter from 8-18 mm, or other transition sizes. In various embodiments, straight portions can have a width or diameter of 8-18 mm, or other sizes. In one embodiment, a distal portion can be tapered. In various embodiments a distal portion can be tapered distally by 1 mm, 0.5 mm or other values. In various embodiments, an intramedullary structure can built to varying lengths, can comprise varying numbers of portions and/or segments (transition, straight, or otherwise) as needed. In various embodiments, lengths can be about 170 mm to about 500 mm.

FIG. 42 illustrates one embodiment of a cross-screw distal end 3034 of an intramedullary structure 3014 that has one or more pre-formed or pre-drilled cross throughbores 3412 for a surgeon to use in securing the distal end in the bone by using one or more bone screws (not shown) through the bone and into one or more cross-holes at various angles to anchor and secure the distal end of the implant in the bone. In various embodiments, bone screw is the same or similar to a locking bolt. In one embodiment bone screw is a locking screw. In one embodiment bone screw is a self-tapping screw. In one embodiment bone screw uses an internal hex interface for driving the screw. In various embodiments bone screw can have a major diameters and lengths and screwing interfaces configured for a particular application. In various embodiments a bone screw has a major diameter of 4 mm, 5 mm, 6 mm, or other diameters. In various embodiments a bone screw has a length in the range of approximately 16 to 120 mm. In one embodiment at least one bone screw is used in at least one throughbore 3412 at the distal end 3034 of the intramedullary structure 3014. In one embodiment at least one bone screw is used in at least one throughbore 3412 near the base 3032 of the intramedullary structure 3014. In various embodiments, one, two, three, four or more through holes 3412 are provided in any intramedullary structure 3014. In one embodiment a throughbore 3412 is a tunnel in an intramedullary structure 3014. In one embodiment throughbore 3412 may merge with another throughbore 3412 to form a multi-conduit pathway. Different throughbores 3412 may be used or optionally provided for options in fixing the device to bone.

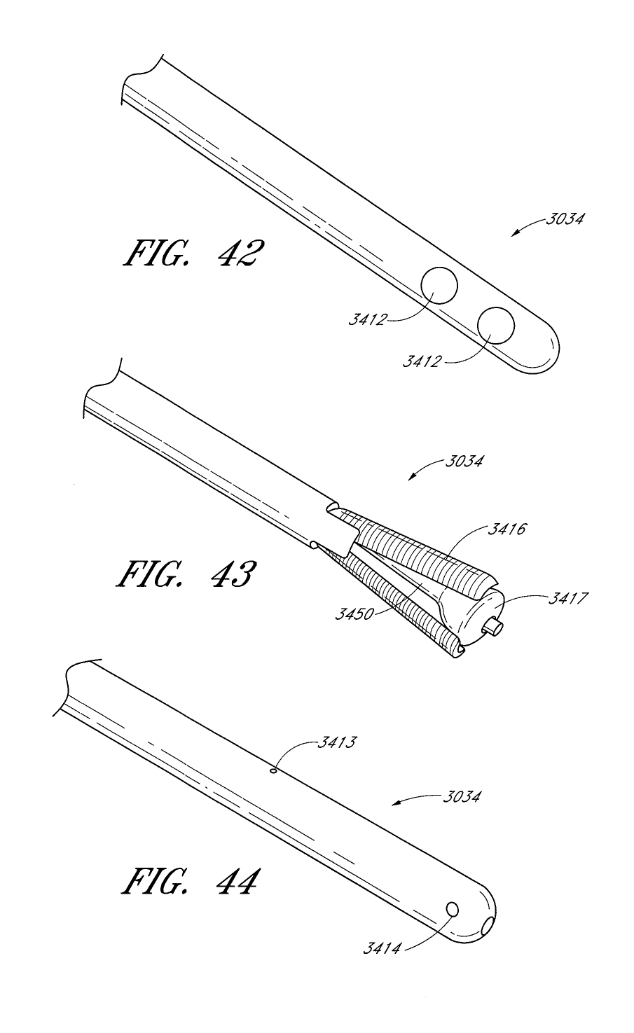

With respect to FIG. 43, one embodiment of a radially-expandable distal end 3034 of an intramedullary structure 3014 can be called “hinged fingers.” In one embodiment, the radially-expandable distal end 3034 is the same or has similar features to an embodiment of the radially-expandable distal end segment described in FIGS. 24-26 of U.S. patent application No. 61/150134, filed Feb. 5, 2009, which is incorporated by reference in its entirety herein. In one embodiment, a radially-expandable distal fixation segment 3415 comprises two or more rigid members 3416 (also called hinged fingers) that can open up like a flower when the ball (or actuator) 3417 at the end of an elongate member 3350 is pulled up proximally through the intramedullary structure 3014. One embodiment includes three or more rigid members 3416. In one embodiment the rigid members 3416 do not bend. One embodiment has metal rigid members 3416. In one embodiment one or more hinged finger members 3416 have a surface texture configured to improve fixation to bone. In one embodiment the surface texture is grooves. In one embodiment, the ball 417 is attached to the distal end of the elongate member 3350. When the elongate member 3350 is pulled proximally toward the base 3032 of the intramedullary structure 300 the ball moves proximally until the hinged fingers 3416 seat on sufficiently stable bone in or around the intramedullary canal. In one embodiment the ball 3417 can move off the central longitudinal axis of the intramedullary device since the elongate member 3350 is flexible, allowing the ball 3417 to apply pressure to actuate the various hinged fingers 416 until a sufficient number of hinged fingers 3416 are properly anchored, irrespective of irregular bony geometry in the intramedullary canal. This self-centering aspect of the ball 3417 and elongate member 3350 is another advantage of the present embodiment.

FIG. 44 illustrates an embodiment of a distal end 3034 of an intramedullary structure 3014 that includes a strong, solid polymer tip. In one embodiment the polymer is implantable-grade polyetheretherketone (PEEK), or other similar materials. One advantage of a polymer distal end 3034 is that the surgeon can pierce the end in any angle or direction to provide cross-screw fixation between the bone and implant. In one embodiment one or more bone screws are used to provide structure between one side of the cortical bone, through the polymer distal end 3034, and into the other side of the cortical bone. In one embodiment the polymer distal end 3034 is fixed to the intramedullary structure 3014 with a pin 3413. In one embodiment the polymer distal end 3034 has one or more markers 3414 placed in it for radiopaque monitoring of the fixation process. In one embodiment the marker 3414 is near a distal end of the polymer distal end 3034. In one embodiment the marker 3414 can be a ring or other structure or shape for visualization under monitoring devices such as fluoroscopy.

The embodiments discussed above have employed three elongate bodies 3016. However, it is understood that more or fewer elongate bodies 3016, and a corresponding number of apertures 3028, may be employed. The embodiments have disclosed using a single intramedullary structure; however other embodiments are contemplated that employ two or more intramedullary structures. Further, although some ways to couple the components of the fixation device to each other and to bone have been expressly disclosed above, such as by a threaded connection, one skilled in the art will recognize that other configurations are possible and are equivalent.

Thus, embodiments of an improved femoral fixation device have been provided as described above. While the structure has been described in terms of certain specific embodiments, there is no intention to limit the invention to the same. Any of the features, shapes, characteristics, materials, capabilities and other aspects of the embodiments of the femoral fixation devices disclosed may be combined or mixed with any of the other embodiments of femoral fixation devices disclosed. Any features, shapes, characteristics, materials, capabilities and other aspects of any embodiment of any anchor, including any intramedullary structure can be used in conjunction with any femoral fixation device. It will be understood that the foregoing is only illustrative of the principles of the invention, and that various modifications, alterations, and combinations can be made by those skilled in the art without departing from the scope and spirit of the invention. Accordingly, it is not intended that the invention be limited, except as by the appended claims.

What is claimed is:

1. An implantable bone fixation structure, comprising:
a plurality of elongate bodies, wherein the elongate bodies each comprise a proximal end and a distal end;
a head portion comprising a plurality of apertures, wherein the apertures are configured to couple to the proximal end of at least one of the elongate bodies;
an intramedullary portion connected to the head portion and configured to fit within an intramedullary canal;
a first configuration comprising a converged position of the elongate bodies, wherein the total distance between the distal ends of the elongate bodies is a first distance; and
a second configuration comprising a diverged position of the elongate bodies, wherein the total distance between the distal ends of the elongate bodies is a second distance, wherein the second distance is greater than the first distance.

2. The bone fixation structure of claim 1, wherein each of the elongate bodies comprises a longitudinal axis, and the longitudinal axes are not co-planar.

3. The bone fixation structure of claim 2, wherein the longitudinal axes of the elongate bodies are not parallel.

4. The bone fixation structure of claim 1, wherein the intramedullary portion is an intramedullary nail.

5. The bone fixation structure of claim 1, wherein the intramedullary portion is segmented.

6. The bone fixation structure of claim 1, wherein the intramedullary portion comprises polyether ether ketone.

7. An implantable femoral fixation device, comprising:
a proximal segment, a distal segment and an intermediate segment disposed between the proximal segment and the distal segment;
a first configuration and a second configuration, the second configuration being larger than the first configuration; and
an anchor.

8. The implantable femoral fixation structure of claim 7, the anchor comprising a segmented intramedullary structure.

9. A method of treating a fracture in a proximal femur, comprising the steps of:
creating an access hole in cortical bone;
creating a pathway in cancellous bone through the femoral neck and into the femoral head;
creating a cavity in cancellous bone between the cavity and a portion of the inside surface of cortical bone in the femoral head;
inserting a femoral fixation device through the access hole; and
anchoring the femoral fixation device to an anchor.

10. The method of claim 9, further comprising the step of transforming the femoral fixation device from a radially reduced configuration to a radially expanded configuration.

11. The method of claim 9, further comprising the steps of:
providing an anchor, having a proximal end and a distal end;
advancing the anchor along a nonlinear path while the anchor is in a flexible state;
engaging the bone with the distal end of the anchor;
transforming the anchor from the flexible state to a substantially rigid state; and
locking the anchor in the substantially rigid state.

12. The method of claim 9, further comprising inserting a fixation media to fill at least a portion of the cavity.

13. The method of claim 9, wherein the femoral fixation device comprises:
a plurality of elongate bodies, wherein the elongate bodies each comprise a proximal end and a distal end;
a head portion comprising a plurality of apertures, wherein the apertures are configured to couple to the proximal end of at least one of the elongate bodies, wherein the head portion is connected to the anchor disposable within an intramedullary canal;
a first configuration comprising a converged position of the elongate bodies, wherein the total distance between the distal ends of the elongate bodies is a first distance; and
a second configuration comprising a diverged position of the elongate bodies, wherein the total distance between the distal ends of the elongate bodies is a second distance, wherein the second distance is greater than the first distance.

14. The method of claim 9, further comprising:
inserting a plurality of elongate bodies through a plurality of apertures in a head portion of the femoral fixation device,
wherein the elongate bodies each comprise a proximal end and a distal end, wherein the apertures are configured to couple to the proximal end of at least one of the elongate bodies,
wherein the head portion is connected to the anchor,
wherein the anchor is disposed within an intramedullary canal,
wherein the plurality of elongate bodies comprises a converged region and a diverged region, the converged region of the elongate bodies comprising a first distance between the elongate bodies, the diverged region of the elongate bodies comprising a second distance between the elongate bodies, wherein the second distance is greater than the first distance.
